SEQUENCE LISTING (4 sequences)

SEQ ID NO: 1 - DNA, 1320 bp, Sorghum bicolor
atgggggaggaggtggtcgtggaggctccgaggcccaagtctcctccgagatacccggacctgtgtgggaggaggaggctgcaactggaggtgcagattctcgatcgcgagatcacgtttctcaaggacgagctgcaactccttgaaggaactcaacctgtttctcgttctggctgctgtaaagaagtcaacgagtctgttggaaccaagcaagacccactcataccaatcaataaaaggaagcacagatcctgccgtctttacttgtggatcagatcaaagttgtgcatatgcatttcatggttctgttgttcctgccaatgtctccctaagtgtaaaggaccaaggtgcttcaattgttcttgttgttcctgctgcgacgagtcaagctgtaggcctaactgctcctcttgttgtgaggagtcttgctgcaagccaaactgctcctcatgctgtgacgagtcatgctgtaggccaaactgcggctcttgctgtgacggttcatgctgcaagacgtcctgctcatgctgttccccaaactgtagctcatgctgcaaacccaactgtagctgcttcaagaccccttcatgctgcaagccaaactgctcatgttgtagcccaaactgtagctcatgctgcaaacccaactgtagctgcttcaagaccccttcatgttgcaagcccaactgttcctgctgttcccctaactgtagctcatgttgcgatacatcttactgcaaaccgaactgtagctgtttcaagatttcttcttgcttcaaatcccagtccagctgctgtggcccaaattgctgcacctgtagccttccgagctgcggctgcgaacctcgttactctactagctgttcctcttgcggtcaatgtaagcccgaatgtggctcatgttctagcaattgctgtgactgcaaaccaagctgttcctgctgtaacgaacagtgctgtagctgcgcagattgctgttcctgctccttccctagatgttccagctgctttagctgtttcaagattttcaaatgctcttgcgccagctgctcaaacttgtgtaagtgctcctgcactcaatgcttcaactgccagtcctcttgttgcaagggacagccttcttgttgcaagtgccagtcatcttgttgtgaagggcaaccttcttgcttcaaaggaggctgttgtcgctcctgctcccttccaaagccgtcatgcccagaatgttcttgtggttgcgtttggtcatgcagaaagtgtacagaaggatgtagatgtcccagatgtcgtaatccgtgctgtacctccggttgcttgtgttga

SEQ ID NO: 2 - protein, 426 aa, Sorghum bicolor
MGEEAVVMEAPRPKSPPRYPDLCGRRRMQLEVQILSREITFLKDELHFLEGAQPVSRSGCIKEINEFVGTKHDPLIPTKRRRHRSCRLFRWIGSKLCICISCLCYCCKCSPKCKRPRCLNCSCSSCCDEPCCKPNCSACCAGSCCSPDCCSCCKPNCSCCKTPSCCKPNCSCSCPSCSSCCDTSCCKPSCTCFNIFSCFKSLYSCFKIPSCFKSQCNCSSPNCCTCTLPSCSCKGCACPSCGCNGCGCPSCGCNGCGCPSCGCNGCGLPSCGCNGCGSCSCAQCKPDCGSCSTNCCSCKPSCNGCCGEQCCRCADCFSCSCPRCSSCFNIFKCSCAGCCSSLCKCPCTTQCFSCQSSCCKRQPSCCKCQSSCCEGQPSCCEGHCCSLPKPSCPECSCGCVWSCKNCTEGCRCPRCRNPCCLSGCLC

SEQ ID NO: 3 - DNA, 639 bp, Sorghum bicolor
atgggagaagaggtggctgctgctgctgccgttgtgctggaaccacctaggcctaagtcaccaccaagataccctgacctctgcgggagaagaaggctccagcttgagcttcaaattcttaacagagaaatcgacttcctgaaggacgaactgcaatctctggagggcgttccacctgtgtctcgcagctgtaaggaagtcaacgagttcgtcggcaccaagcaagatcccttgttgcccattaagaaaaagacgcatcgttcatgcagacttttctggtggatcagaagcaagttgtgcatatgcgtttcctggttctgttgctcatgccactgtctccctaattgtaagagaccatgctgcttggattgctcctgctgttcctgtccagacttgtcatgttgcaaaccttcttgcaaatcctgcaataaaccctgctcttggccgaacagctgttcttgctgtgatacaccgtgttgtaagccggattgtccctcatgttcttcatcctgctccagctgctgtaacctttcttgctgcaacccgaactgctccagctgctgcacttgcaacccgtcttgctgtaagccaaattgcaatagctgctgtcgtcctaattgctcaagctgttgcaactga

SEQ ID NO: 4 - protein, 212 aa, Sorghum bicolor
MGEEVAAAAAVVLEPPRPKSPPRYPDLCGRRRLQLELQILNREIDFLKDELQSLEGVPPVSRSCKEVNEFVGTKQDPLLPIKKKTHRSCRLFWWIRSKLCICVSWFCCSCHCLPNCKRPCCLDCSCCSCPDLSCCKPSCKSCNKPCSWPNSCSCCDTPCCKPDCPSCSSSCSSCCNLSCCNPNCSSCCTCNPSCCKPNCNSCCRPNCSSCCN

(19) United States
(12) Patent Application Publication
(10) Pub. No.: US 2017/0096677 A1
(43) Pub. Date: Apr. 6, 2017

(54) GENETICALLY MODIFIED PLANTS FOR CROP YIELD ENHANCEMENT

(71) Applicant: Dow AgroSciences LLC, Indianapolis, IN (US)

(72) Inventors: Narasimha Chary Samboju, Carmel, IN (US); Weiting Ni, Carmel, IN (US); John Davies, Indianapolis, IN (US)

(73) Assignee: Dow AgroSciences LLC, Indianapolis, IN (US)

(21) Appl. No.: 15/281,872

(22) Filed: Sep. 30, 2016

Related U.S. Application Data
(60) Provisional application No. 62/237,151, filed on Oct. 5, 2015.

(51) Int. Cl. C12N 15/82; C07K 14/415

(52) U.S. Cl. CPC C12N 15/8261; C07K 14/415; C12N 15/8205

(57) ABSTRACT

This invention is related to methods for producing a plant having improved/enhanced crop yield as compared to a corresponding wild type plant, such method comprising overexpression of a qPE9-1 gene and/or a Dense and Erect Panicle 1 (DEP1) gene. Provided are nucleic acids encoding for qPE9-1 and/or DEP1, and cells, progenies, seeds and pollen derived from such plants or parts, as well as methods of making and methods of using such plant cell(s) or plant(s), progenies, seed(s) or pollen. This invention relates generally to a crop plant with increased yield, preferably under condition of transient and repetitive stress as compared to a corresponding non-transformed wild type plant cell. This invention is also related to methods of producing and screening for and breeding such crop plants or plant cells.

### CROSS REFERENCE TO RELATED APPLICATION

This application claims priority under 35 U.S.C. §119 of U.S. Provisional Patent Application Ser. No. 62/237,151 filed Oct. 5, 2015, which is hereby incorporated by reference in its entirety.

### FIELD OF THE INVENTION

This invention is generally related to the field of agriculture, and more specifically the field of transgenic plants with fusion proteins for crop yield enhancement.

### BACKGROUND OF THE INVENTION

Population increases and climate change have brought the possibility of global food, feed, and fuel shortages into sharp focus in recent years. Under field conditions, plant performance, for example in terms of growth, development, biomass accumulation and seed generation, depends on a plant's tolerance and acclimation ability to numerous environmental conditions, changes and stresses. Since the beginning of agriculture and horticulture, there is a need for improving plant traits in crop cultivation. Breeding strategies foster crop properties to withstand biotic and abiotic stresses, to improve nutrient use efficiency and to alter other intrinsic crop specific yield parameters, i.e., increasing yield by applying technical advances.

A quantitative trait phenotype is presumed to be caused by the simultaneous segregation of many genes, each of which contributes a small, additive effect on the phenotype, and the interaction of the genotype with the environment. Quantitative trait loci (QTLs) may be identified that are DNA segments (e.g., markers) linked to the quantitative trait. Once several QTLs are identified for a trait, it may be easier to model the expression of the trait as a single mathematical function, rather than to explicitly consider each individual locus. In addition, the QTLs identified for a particular trait are often specific to the parental genotypes used in the mapping cross. For example, the contribution of a gene to a quantitative trait that is important in one genotype may not be so in another, where the gene's contribution is not observable in the absence of a second gene that is differentially expressed in the two genotypes.

Grain yield in corn is a complex, quantitative trait influenced by genetic, environmental, and management factors. Similar to other crops, grain yield in corn can be dissected into a number of key yield components including kernel number per plant and weight per kernel. While genetic- and agronomic-based improvement of either of these components can result in enhanced grain production, kernel number is more impacted by greater resource availability and/or improved genetics than kernel weight and thus likely presents a more robust avenue for grain yield improvement

Thus, there remains a need to enhance grain yield of various crops including corn.

### SUMMARY OF THE INVENTION

This invention is related to methods for producing a plant having improved/enhanced crop yield as compared to a corresponding wild type plant, such method comprising overexpression of a qPE9-1 gene and/or a Dense and Erect Panicle 1 (DEP1) gene. Provided are nucleic acids encoding for qPE9-1 and/or DEP1, and cells, progenies, seeds and pollen derived from such plants or parts, as well as methods of making and methods of using such plant cell(s) or plant(s), progenies, seed(s) or pollen. This invention relates generally to a crop plant with increased yield, preferably under condition of transient and repetitive stress as compared to a corresponding non-transformed wild type plant cell. This invention is also related to methods of producing and screening for and breeding such crop plants or plant cells.

In one aspect, provided is a plant transformation vector comprising a nucleic acid encoding a polypeptide having at least 80%, 85%, 88%, 90%, 92%, 95%, 98%, 99%, or 100% sequence identity to SEQ ID NO: 2 or 4. In one embodiment, the nucleic acid is operably linked to a constitutive promoter. In another embodiment, the nucleic acid has at least 80%, 85%, 88%, 90%, 92%, 95%, 98%, 99%, or 100% sequence identity to SEQ ID NO: 1 or 3.

In another aspect, provided is a nucleic acid construct for transgenic plants. The nucleic acid construct comprises (a) a polynucleotide sequence encoding a polypeptide having at least 80% sequence identity to SEQ ID NO: 2 or 4; and (b) one or more control sequences for driving expression of the polynucleotide sequence in the transgenic plants. In some embodiments, the polynucleotide sequence is codon optimized for expression in the transgenic plants.

In one embodiment, the plants are monocotyledons plants. In another embodiment, the plants are dicotyledons plants. In another embodiment, the plants are not monocotyledons plants. In another embodiment, the plants are not dicotyledons plants. In one embodiment, the nucleic acid construct is stably transformed into the transgenic plants. In another embodiment, the nucleic acid construct comprises a binary vector for Agrobacterium-mediated transformation. In one embodiment, the nucleic construct comprises a selectable marker. In a further embodiment, the selectable marker is an aryloxyalkanoate dioxygenase. In a further embodiment, the aryloxyalkanoate dioxygenase is AAD-1 or AAD-12.

In one embodiment, the polynucleotide sequence has at least 80%, 85%, 88%, 90%, 92%, 95%, 98%, 99%, or 100% identity to SEQ ID NO: 1 or 3. In another embodiment, the one or more control sequences comprise a viral sequence. In another embodiment, the one or more control sequences comprise a plant promoter. In another embodiment, the one or more control sequences do not comprise a viral sequence. In another embodiment, the one or more control sequences do not comprise a plant promoter.

In another aspect, provided is a method for producing a transgenic plant. The method comprises introducing into the plant a heterologous nucleic acid encoding a polypeptide having at least 80%, 85%, 88%, 90%, 92%, 95%, 98%, 99%, or 100% sequence identity to SEQ ID NO: 1 or 3. In one embodiment, the heterologous nucleic acid is introduced into the plant by Agrobacterium-mediated transformation.

In another aspect, provided is a method for enhancing crop yield. The method comprises:
(a) transforming a plant cell with the plant transformation vector or nucleic acid construct provided herein;
(b) regenerating the transformed plant cell into a transgenic plant; and
(c) planting the transgenic plant in a crop field.

In one embodiment of the method provided, there is statistically significant improvement of at least one parameter selected from the group consisting of grain yield per hectare (GYH), grain yield per plant (GPP), averaged grain size (AGS), kernel number per plant (KPP), and averaged kernel weight (AKW) as compared to negative control plant. In another embodiment, there are statistically significant improvements of at least two parameters selected from the group consisting of grain yield per hectare (GYH), grain yield per plant (GPP), averaged grain size (AGS), kernel number per plant (KPP), and averaged kernel weight (AKW). In another embodiment, there are statistically significant improvements of at least three parameters selected from the group consisting of grain yield per hectare (GYH), grain yield per plant (GPP), averaged grain size (AGS), kernel number per plant (KPP), and averaged kernel weight (AKW). In another embodiment, there are statistically significant improvements of four parameters selected from the group consisting of grain yield per hectare (GYH), grain yield per plant (GPP), averaged grain size (AGS), kernel number per plant (KPP), and averaged kernel weight (AKW). In another embodiment, the enhanced crop yield is at least 5%, 8%, 10%, 12%, 15%, 20%, 25%, 30%, 50%, or 100%.

In another aspect, provided is a transgenic plant generated from the method provided herein. In one embodiment, the plant is Zea mays or Glycine max. In another aspect, provided is a plant comprising a heterologous nucleic acid encoding a polypeptide having at least 80%, 85%, 88%, 90%, 92%, 95%, 98%, 99%, or 100% sequence identity to a polypeptide selected from the group consisting of SEQ ID NO: 2 or 4. In one embodiment, there is statistically significant improvement of at least one parameter selected from the group consisting of grain yield per hectare (GYH), grain yield per plant (GPP), averaged grain size (AGS), kernel number per plant (KPP), and averaged kernel weight (AKW) as compared to negative control plant. In another embodiment, there are statistically significant improvements of at least two parameters selected from the group consisting of grain yield per hectare (GYH), grain yield per plant (GPP), averaged grain size (AGS), kernel number per plant (KPP), and averaged kernel weight (AKW). In another embodiment, there are statistically significant improvements of at least three parameters selected from the group consisting of grain yield per hectare (GYH), grain yield per plant (GPP), averaged grain size (AGS), kernel number per plant (KPP), and averaged kernel weight (AKW). In another embodiment, there are statistically significant improvements of four parameters selected from the group consisting of grain yield per hectare (GYH), grain yield per plant (GPP), averaged grain size (AGS), kernel number per plant (KPP), and averaged kernel weight (AKW). In another embodiment, the enhanced crop yield is at least 5%, 8%, 10%, 12%, 15%, 20%, 25%, 30%, 50%, or 100%.

In another aspect, provided is a process for producing a plant, plant seed, or progeny thereof. The method comprises:
(a) transforming a plant cell with the plant transformation vector or the nucleic acid construct provided herein;
(b) growing a plant from the transformed plant cell until the plant produces seed; and
(c) harvesting the seed from the plant.

In another aspect, provided is a seed harvested from the plant produced by the process provided herein. In another aspect, provided is a genetically transformed plant or seed, characterized in that its genome has been transformed to contain the plant transformation vector or the nucleic acid construct provided herein.

In one embodiment, there is statistically significant improvement of at least one parameter selected from the group consisting of grain yield per hectare (GYH), grain yield per plant (GPP), averaged grain size (AGS), kernel number per plant (KPP), and averaged kernel weight (AKW) as compared to its non-transgenic parent plant or seed. In another embodiment, there are statistically significant improvements of at least two parameters selected from the group consisting of grain yield per hectare (GYH), grain yield per plant (GPP), averaged grain size (AGS), kernel number per plant (KPP), and averaged kernel weight (AKW). In another embodiment, there are statistically significant improvements of at least three parameters selected from the group consisting of grain yield per hectare (GYH), grain yield per plant (GPP), averaged grain size (AGS), kernel number per plant (KPP), and averaged kernel weight (AKW). In another embodiment, there are statistically significant improvements of four parameters selected from the group consisting of grain yield per hectare (GYH), grain yield per plant (GPP), averaged grain size (AGS), kernel number per plant (KPP), and averaged kernel weight (AKW). In another embodiment, the enhanced crop yield is at least 5%, 8%, 10%, 12%, 15%, 20%, 25%, 30%, 50%, or 100%.

### BRIEF DESCRIPTION OF THE DRAWINGS

FIG. 1A shows exemplary nitrogen (N) treatment levels and grain yield responses averaged across all constructs (and their respective events) at Location 1 and Location 2 tested.

FIG. 1B shows exemplary grain yield coefficient of variation (CV) values and nitrogen (N) treatment levels at Location 1 and Location 2 tested. The dashed red line indicates the 10% grain yield CV threshold above which CV values are undesirably high.

FIG. 2A shows exemplary grain yield differences between selected events for the pDAB 111974 construct tested. Numbers above each vertical bar indicate grain yield difference values (%) from their respective negative controls. Results shown are based on a standard t-test comparison. *Statistically significant at p=0.10. **Statistically significant at p=0.05. ***Statistically significant at p=0.01. NS, not statistically significant at p=0.10.

FIG. 2B shows exemplary grain yield differences between selected events for the pDAB111975 construct tested. Numbers above each vertical bar indicate grain yield difference values (%) from their respective negative controls. Results shown are based on a standard t-test comparison. *Statistically significant at p=0.10. **Statistically significant at p=0.05. ***Statistically significant at p=0.01. NS, not statistically significant at p=0.10.

FIG. 3A shows a representative map of plasmid pDAB111974. FIG. 3B shows a representative map of plasmid pDAB111975. Both plasmids comprise the qPE9-1 sequence.

FIG. 4A shows a representative map of plasmid pDAB110459. FIG. 4B shows a representative map of plasmid pDAB109263. Both plasmids comprise the SbDEP1 sequence.

FIG. 5 shows exemplary daily precipitation, daily evapotranspiration, and accumulated growing degree day (GDD) information for the Location 1 and Location 2 tested.

FIG. 6 shows additional exemplary nitrogen (N) treatment levels and grain yield responses averaged across all constructs (and their respective events) at Locations 1, 2 and 3 tested.

FIG. 7A shows additional exemplary grain yield differences between selected events for the pDAB111974 construct tested. Numbers above each vertical bar indicate grain yield difference values (%) from their respective negative controls. Results shown are based on a standard t-test comparison. *Statistically significant at p=0.10. **Statistically significant at p=0.05. ***Statistically significant at p=0.01. NS, not statistically significant at p=0.10.

FIG. 7B shows additional exemplary grain yield differences between selected events for the pDAB111975 construct tested. Numbers above each vertical bar indicate grain yield difference values (%) from their respective negative controls. Results shown are based on a standard t-test comparison. *Statistically significant at p=0.10. **Statistically significant at p=0.05. ***Statistically significant at p=0.01. NS, not statistically significant at p=0.10.

### DETAILED DESCRIPTION OF THE INVENTION

The DENSE AND ERECT PANICLE1 (DEP1) locus in rice (Oryza sativa L.) is pleiotropically responsible for high panicle density, improved grain number per panicle, and enhanced panicle erectness. Through its impacts on these traits, it often enhances rice grain yield. A functionally equivalent allele to the dominant DEP1 allele in rice is present in various small-grain cereals. As provided herein, yield enhancement in corn (Zea mays L.) and other crops through transgenic approaches can be achieved by introduction of DEP1 and/or qPE9-1, and/or additional dominant negative variants of DEP1. Also provided are plant transformation vectors/nucleic acid constructs containing a bamboo (Phyllostachys heterocycla) DEP1 ortholog BpqPE9 (ZmUbi1::BpqPE9 and ZmGZein27::BpqPE9).

For the first construct, the BpqPE9 gene is driven by the ZmUbi1 promotor in an attempt to mimic the impacts of the dominant rice DEP1 allele on plant architecture, N response, and grain yield. For the second construct, the BpqPE9 gene is driven by the ZmGZein27 promotor to examine the effects of this ortholog on grain yield alone. Effects are evaluated on genetic yield potential, N responsiveness, and N stress tolerance via the grain yield responses of B104×LLH37 corn plants. Selected events for the ZmUbi1::BpqPE9 and ZmGZein27::BpqPE9 constructs show significantly positive enhancements on grain yield, where such enhancements generally range from 4 to 10%, but some event-level impacts are markedly higher.

Unless otherwise indicated, all technical and scientific terms used herein have the same meaning as they would to one skilled in the art of the present invention. Practitioners are particularly directed to Sambrook et al. Molecular Cloning: A Laboratory Manual (Second Edition), Cold Spring Harbor Press, Plainview, N.Y., 1989, and Ausubel F M et al. Current Protocols in Molecular Biology, John Wiley & Sons, New York, N.Y., 1993, for definitions and terms of the art. It is to be understood that this invention is not limited to the particular methodology, protocols, and reagents described, as these may vary.

In some embodiments, genes encoding such a polypeptide may be utilized to over-express or reduce the expression of the polypeptide in a plant in which the polypeptide may normally be found. For example, the gene or an equivalent thereof may be introduced into a cell of the plant in a genetic locus where the gene is not normally found, or a native copy of the gene may be placed under regulatory control elements that lead to increased expression of the native gene. In other examples, the gene may be used to design a polynucleotide that inhibits the expression of the gene, and the polynucleotide may be introduced into a cell of the plant.

### ABBREVIATIONS

CAI codon adaptation index

dsRNA double-stranded ribonucleic acid

hpRNA hairpin ribonucleic acid

iRNA inhibitory ribonucleic acid

MILC measure independent of length and composition

miRNA micro inhibitory ribonucleic acid

ORF open reading frame

PCR polymerase chain reaction

QTL quantitative trait locus

RBS ribosome binding site

RISC RNA-induced Silencing Complex

RNAi ribonucleic acid interference

RSCU relative synonymous codon usage

RT-PCR real-time PCR

siRNA small inhibitory ribonucleic acid

TRP transpiration reduction point

Use of the singular forms “a,” “an,” and “the” include plural references, unless the context clearly dictates otherwise. For example, reference to “a polynucleotide” includes a plurality of polynucleotides, reference to “a substrate” includes a plurality of such substrates, reference to “a variant” includes a plurality of such variants, etc.

Where a range of values is recited, each intervening integer value, and each fraction thereof, between the recited upper and lower limits of that range is also specifically disclosed, along with each sub-range between such values. The upper and lower limits of any range can independently be included in, or excluded from, the range, and each range where either, neither, or both limits are included is also encompassed. Where a value being discussed has inherent limits (for example, where a component can be present at a concentration of from 0 to 100%, or where the pH of an aqueous solution can range from 1 to 14), those inherent limits are specifically disclosed.

Where a value is explicitly recited, values that are about the same quantity or amount as the recited value are also within the scope of the value. Where a combination is disclosed, each subcombination of the elements of that combination is also specifically included. Conversely, where different elements or groups of elements are individually disclosed, combinations thereof are also disclosed. Where any element or aspect of an embodiment is disclosed as having a plurality of alternatives, examples of that element or aspect in which each alternative is excluded singly, or in any combination with the other alternatives, are also hereby disclosed.

Unless otherwise provided, all technical and scientific terms used herein have the same meaning as commonly understood by one of ordinary skill in the art of genetics, bioinformatics, and gene design. General dictionaries containing many of the terms used in this disclosure are: Singleton et al. (1994) Dictionary of Microbiology and Molecular Biology, 2nd Ed., John Wiley and Sons, New York; and Hale and Marham (1991) The Harper Collins Dictionary of Biology, Harper Perennial, New York. Any methods and materials similar or equivalent to those described herein may be used in the practice or testing of embodiments, though certain methods and materials are exemplified by those explicitly recited herein.

As used herein, the phrase “vector” refers to a piece of DNA, typically double-stranded, which can have inserted into it a piece of foreign DNA. The vector can be for example, of plasmid or viral origin, which typically encodes a selectable or screenable marker or transgenes. The vector is used to transport the foreign or heterologous DNA into a suitable host cell. Once in the host cell, the vector can replicate independently of or coincidental with the host chromosomal DNA. Alternatively, the vector can target insertion of the foreign or heterologous DNA into a host chromosome.

As used herein, the phrase “transgene vector” refers to a vector that contains an inserted segment of DNA, the “transgene” that is transcribed into mRNA or replicated as a RNA within a host cell. The phrase “transgene” refers not only to that portion of inserted DNA that is converted into RNA, but also those portions of the vector that are necessary for the transcription or replication of the RNA. A transgene typically comprises a gene-of-interest but needs not necessarily comprise a polynucleotide sequence that contains an open reading frame capable of producing a protein.

As used herein, the phrase “transformed” or “transformation” refers to the introduction of DNA into a cell. The phrases “transformant” or “transgenic” refers to plant cells, plants, and the like that have been transformed or have undergone a transformation procedure. The introduced DNA is usually in the form of a vector containing an inserted piece of DNA.

As used herein, the phrase “transgenic plant” refers to a plant whose genome has been altered by the stable integration of recombinant DNA. A transgenic plant includes a plant regenerated from an originally-transformed plant cell and progeny transgenic plants from later generations or crosses of a transformed plant.

As used herein, the phrase “recombinant DNA” refers to DNA which has been genetically engineered and constructed outside of a cell including DNA containing naturally occurring DNA or cDNA or synthetic DNA.

As used herein, the term “gene expression” refers to the process by which the coded information of a nucleic acid transcriptional unit (e.g., an ORF) is converted into an operational, non-operational, or structural part of a cell, for example, via the synthesis of an encoded protein. Gene expression can be measured at the RNA level, or the protein level for translated expression products, by methods, including, for example and without limitation, Northern blot, RT-PCR, Western blot, or in vitro, in situ, or in vivo protein activity assay(s).

As used herein with respect to a specified nucleic acid, the term “encoding” or “encoded” refers to the process for translation of a polynucleotide into the specified structural part of a cell (e.g., protein). A polynucleotide encoding a protein may comprise intervening sequences (e.g., introns) within translated regions of the nucleic acid, or may lack such intervening non-translated sequences (e.g., cDNA). The information by which a protein is encoded is specified by the use of codons. Typically, the amino acid sequence is encoded by the nucleic acid using the “universal” genetic code. However, variants of the universal code, such as are present in some plant, animal and fungal mitochondria, the bacterium Mycoplasma capricolumn or the ciliate Macronucleus, may be used when the polynucleotide is expressed therein.

When a polynucleotide is prepared or altered synthetically, advantage can be taken of known codon preferences of the intended host where the polynucleotide is to be expressed. For example, although polynucleotides may be expressed in some embodiments in both monocotyledonous and dicotyledonous plant species, a polynucleotide sequence may be modified (e.g., optimized) to account for the specific codon preferences and GC content preferences of monocots or dicots. See, e.g., Murray et al. (1989) Nucl. Acids Res. 17:477-98 (Maize codon usage for 28 genes from maize plants).

As used herein, the term “full-length” sequence refers to the entire amino acid sequence of a biologically-active form of a specified protein, or to the entire coding nucleotide sequence of a polynucleotide encoding such a protein. Methods to determine whether a sequence is full-length include, for example and without limitation, Northern or Western blots, primer extension, S1 protection, and ribonuclease protection. See, e.g., Plant Molecular Biology: A Laboratory Manual, Clark, Ed., Springer-Verlag, Berlin (1997). Comparison to known full-length homologous, orthologous, and/or paralogous sequences may also be used to identify a full-length sequence. Additionally, consensus sequences typically present at the 5′ and 3′ untranslated regions of mRNA may aid in the identification of a polynucleotide as full-length.

As used herein, the term “codon usage bias,” or simply “codon usage,” refers to the high-frequency preferential use of a particular codon (as opposed to other, synonymous codons) coding for an amino acid within an organism. A codon usage bias may be expressed as a quantitative measurement of the rate at which a particular codon is used in the genome of a particular organism, for example, when compared to other codons that encode the same amino acid.

Various methods are known to those of skill in the art for determining codon usage bias. In some embodiments, codon usage bias may be determined by the CAI method, which is essentially a measurement of the distance of a gene's codon usage to the codon usage of a predefined set of highly-expressed genes. Sharp and Li (1987) Nucleic Acids Res. 15:1281-95. Alternative methods for determining a codon usage bias include AMC (Supek and Vlahovicek (2005) BMC Bioinformatics 6:182) and RSCU, which is the observed frequency of a particular codon divided by the frequency expected from equal usage of all the synonymous codons for that amino acid (Sharp et al. (1986) Nucleic Acids Res. 14:5125-43). RSCU values close to 1.0 indicate a lack of bias for the particular codon, whereas departure from 1.0 reflects codon usage bias.

Thus, codon usage bias includes the relative frequencies of use of codons that encode the same amino acid (“synonymous codons”). A bias may be naturally occurring; for example, where the codon bias in an organism's genome reflects the relative overall use of synonymous codons within all the genes in that organism. A bias may also be used in a computational algorithm, where, for example, it may be used to determine the relative frequency with which different synonymous codons are selected for use in designing a polynucleotide sequence. Similarly, the “relative” frequency of any sequence element used to encode a polypeptide within a nucleotide sequence is the frequency with which that sequence element is used to encode a feature (e.g., amino acid, amino acid pair, etc.) of the polypeptide, divided by the number of occurrences within the polypeptide in a given reading frame of features that could be encoded by that sequence element.

Codon usage bias may also be inferred from a codon usage table for a particular expression host organism. Codon usage tables are readily available for many expression host organisms. See, e.g., Nakamura et al. (2000) Nucleic Acids Res. 28:292 (Codon Usage Database—updated versions available at kazusa.or.jp/codon). When a codon usage table is not available, it may be assembled from public organismal genetic databases, such as those maintained by NCBI (available at ncbi.nlm.nih.gov/sites/genome). In some embodiments, a codon usage table may be assembled from a set of coding regions obtained from the particular expression host organism. In some examples, a set of coding regions comprises at least 100, at least 200, at least 300, at least 400, at least 500, at least 550, at least 600, or more coding regions obtained from the particular expression host organism.

The terms “codon usage table,” or “codon bias table,” or “codon frequency table” are used interchangeably to describe a table which correlates each codon that may be used to encode a particular amino acid with the frequencies with which each codon is used to encode that amino acid in a specific organism, within a specified class of genes within that organism, or within one or more synthetic polynucleotides.

As used herein, the term “absolute codon frequency” refers to the frequency with which a codon appears relative to the total number of codons (e.g., both synonymous and non-synonymous codons) within a polynucleotide or set of polynucleotides in a given reading frame (e.g., a reading frame that is used to encode a polypeptide of interest). Similarly, the “absolute” frequency of any sequence element used to encode a polypeptide within a polynucleotide is the frequency with which that sequence element is used to encode a feature of the polypeptide, divided by the number of occurrences within the polypeptide of features of the same size as those that could be encoded by that sequence element.

As used herein, the term “codon space” refers to all of the possible polynucleotide sequences that can be used to encode a specific polypeptide, by varying the codons used to encode amino acids within the polypeptide.

Codon substitution: As used herein, the term “codon substitution” refers to the altering of a nucleotide coding sequence by changing one or more of the codons encoding one or more amino acids of an encoded polypeptide, without altering the amino acid sequence of the encoded polypeptide.

Codon optimization: As used herein, the term “codon optimization” refers to processes employed to modify an existing coding sequence, or to design a coding sequence in the first instance, for example, to improve translation in an expression host cell or organism of a transcript RNA molecule transcribed from the coding sequence, or to improve transcription of a coding sequence. Codon optimization includes, but is not limited to, processes including selecting codons for the coding sequence to suit the codon preference of an expression host organism. Codon optimization also includes, for example, the process sometimes referred to as “codon harmonization,” wherein codons of a codon sequence that are recognized as low-usage codons in the source organism are altered to codons that are recognized as low-usage in the new expression host. This process may help expressed polypeptides to fold normally by introducing natural and appropriate pauses during translation/extension. Birkholtz et al. (2008) Malaria J. 7:197-217.

As used herein to describe an effect on a coding polynucleotide (e.g., a gene), the term “inhibition” refers to a measurable decrease in the cellular level of mRNA transcribed from the coding polynucleotide, and/or in the cellular level of a peptide, polypeptide, or protein product of the coding polynucleotide. In some examples, expression of a coding sequence may be inhibited, such that expression is substantially eliminated. “Specific inhibition” refers to the inhibition of a target coding polynucleotide without consequently affecting expression of other coding polynucleotides in a cell wherein the specific inhibition is being accomplished.

An “isolated” biological component (such as a nucleic acid or protein) has been substantially separated, produced apart from, or purified away from other biological components in the cell of the organism in which the component naturally occurs (i.e., other chromosomal and extra-chromosomal DNA and RNA, and proteins), while effecting a chemical or functional change in the component (e.g., a nucleic acid may be isolated from a chromosome by breaking chemical bonds connecting the nucleic acid to the remaining DNA in the chromosome). Nucleic acid molecules and proteins that have been “isolated” include nucleic acid molecules and proteins purified by standard purification methods. The term also embraces nucleic acids and proteins prepared by recombinant expression in a host cell, as well as chemically-synthesized nucleic acid molecules, proteins, and peptides.

As used herein, the term “nucleic acid molecule” may refer to a polymeric form of nucleotides, which may include both sense and anti-sense strands of RNA, cDNA, genomic DNA, and synthetic forms and mixed polymers of the above. A nucleotide may refer to a ribonucleotide, deoxyribonucleotide, or a modified form of either type of nucleotide. A “nucleic acid molecule” as used herein is synonymous with “nucleic acid” and “polynucleotide.” A nucleic acid molecule is usually at least 10 bases in length, unless otherwise specified. The term includes single- and double-stranded forms of DNA. A nucleic acid molecule can include either or both naturally occurring and modified nucleotides linked together by naturally occurring and/or non-naturally occurring nucleotide linkages.

Nucleic acid molecules may be modified chemically or biochemically, or may contain non-natural or derivatized nucleotide bases, as will be readily appreciated by those of skill in the art. Such modifications include, for example, labels, methylation, substitution of one or more of the naturally occurring nucleotides with an analog, internucleotide modifications (e.g., uncharged linkages: for example, methyl phosphonates, phosphotriesters, phosphoramidates, carbamates, etc.; charged linkages: for example, phosphorothioates, phosphorodithioates, etc.; pendent moieties: for example, peptides; intercalators: for example, acridine, psoralen, etc.; chelators; alkylators; and modified linkages: for example, alpha anomeric nucleic acids, etc.). The term “nucleic acid molecule” also includes any topological conformation, including single-stranded, double-stranded, partially duplexed, triplexed, hairpinned, circular, and padlocked conformations.

The term “exogenous,” as applied to nucleic acids (e.g., polynucleotides, DNA, RNA, and genes) herein, refers to one or more nucleic acid(s) that are not normally present within their specific environment or context. For example, if a host cell is transformed with a nucleic acid that does not occur in the untransformed host cell in nature, then that nucleic acid is exogenous to the host cell. The term exogenous, as used herein, also refers to one or more nucleic acid(s) that are identical in sequence to a nucleic acid already present in a host cell, but that are located in a different cellular or genomic context than the nucleic acid with the same sequence already present in the host cell. For example, a nucleic acid that is integrated in the genome of the host cell in a different location than a nucleic acid with the same sequence is normally integrated in the genome of the host cell is exogenous to the host cell. Furthermore, a nucleic acid (e.g., a DNA molecule) that is present in a plasmid or vector in the host cell is exogenous to the host cell when a nucleic acid with the same sequence is only normally present in the genome of the host cell.

The term “heterologous,” as applied to nucleic acids (e.g., polynucleotides, DNA, RNA, and genes) herein, means of different origin. For example, if a host cell is transformed with a nucleic acid that does not occur in the untransformed host cell in nature, then that nucleic acid is heterologous (and exogenous) to the host cell. Furthermore, different elements (e.g., promoter, enhancer, coding sequence, terminator, etc.) of a transforming nucleic acid may be heterologous to one another and/or to the transformed host. The term heterologous, as used herein, does not include nucleic acids that are identical in sequence to a nucleic acid already present in a host cell, but that are now linked to different additional sequences and/or are present in a different location in the genome.

Marker-assisted breeding: As used herein, the term “marker-assisted breeding” may refer to an approach to breeding directly for one or more trait(s) (e.g., a polygenic trait) and/or QTLs. Marker-assisted breeding provides a time- and cost-efficient process for improvement of plant varieties. In current practice, plant breeders attempt to identify easily detectable traits, such as flower color, seed coat appearance, or isozyme variants, which are linked to an agronomically desired trait. The plant breeders then follow the agronomic trait in the segregating, breeding populations by following the segregation of the easily detectable trait. However, there are very few of these linkage relationships between traits of interest and easily detectable traits available for use in plant breeding. In some embodiments of the invention, marker-assisted breeding comprises identifying one or more genetic markers that are linked to a trait of interest or QTL, and following the trait of interest or QTL in a segregating, breeding population by following the segregation of the one or more genetic markers. In some examples, the segregation of the one or more genetic markers may be determined utilizing a probe for the one or more genetic markers by assaying a genetic sample from a progeny plant for the presence of the one or more genetic markers.

As used herein, the term “conservative substitution” refers to a substitution where an amino acid residue is substituted for another amino acid in a same amino acid structural class. A non-conservative amino acid substitution is one where the residues do not fall into the same structural class (for example, substitution of a basic amino acid for a neutral or non-polar amino acid). Classes of amino acids that may be defined for the purpose of performing a conservative substitution are aliphatic amino acids (e.g., hydrophobic aliphatic amino acids); aromatic amino acids (e.g., uncharged aromatic amino acids); hydrophobic amino acids (e.g., non-aromatic hydrophobic amino acids); polar amino acids (e.g., positively charged polar amino acids, negatively charged polar amino acids, and uncharged polar amino acids); electrically neutral amino acids; and non-polar amino acids.

In some embodiments, a conservative substitution includes the substitution of a first aliphatic amino acid for a second, different aliphatic amino acid. For example, if a first amino acid is one of Gly; Ala; Pro; Be; Leu; Val; and Met, the first amino acid may be replaced by a second, different amino acid selected from Gly; Ala; Pro; Be; Leu; Val; and Met. In particular examples, if a first amino acid is one of Gly; Ala; Pro; Be; Leu; and Val, the first amino acid may be replaced by a second, different amino acid selected from Gly; Ala; Pro; Be; Leu; and Val. In particular examples involving the substitution of hydrophobic aliphatic amino acids, if a first amino acid is one of Ala; Pro; Be; Leu; and Val, the first amino acid may be replaced by a second, different amino acid selected from Ala; Pro; Be; Leu; and Val.

In some embodiments, a conservative substitution includes the substitution of a first aromatic amino acid for a second, different aromatic amino acid. For example, if a first amino acid is one of His; Phe; Trp; and Tyr, the first amino acid may be replaced by a second, different amino acid selected from His; Phe; Trp; and Tyr. In particular examples involving the substitution of uncharged aromatic amino acids, if a first amino acid is one of Phe; Trp; and Tyr, the first amino acid may be replaced by a second, different amino acid selected from Phe; Trp; and Tyr.

In some embodiments, a conservative substitution includes the substitution of a first hydrophobic amino acid for a second, different hydrophobic amino acid. For example, if a first amino acid is one of Ala; Val; Be; Leu; Met; Phe; Tyr; and Trp, the first amino acid may be replaced by a second, different amino acid selected from Ala; Val; Be; Leu; Met; Phe; Tyr; and Trp. In particular examples involving the substitution of non-aromatic, hydrophobic amino acids, if a first amino acid is one of Ala; Val; Be; Leu; and Met, the first amino acid may be replaced by a second, different amino acid selected from Ala; Val; Be; Leu; and Met.

In some embodiments, a conservative substitution includes the substitution of a first polar amino acid for a second, different polar amino acid. For example, if a first amino acid is one of Ser; Thr; Asn; Gln; Cys; Gly; Pro; Arg; His; Lys; Asp; and Glu, the first amino acid may be replaced by a second, different amino acid selected from Ser; Thr; Asn; Gln; Cys; Gly; Pro; Arg; His; Lys; Asp; and Glu. In particular examples involving the substitution of uncharged, polar amino acids, if a first amino acid is one of Ser; Thr; Asn; Gln; Cys; Gly; and Pro, the first amino acid may be replaced by a second, different amino acid selected from Ser; Thr; Asn; Gln; Cys; Gly; and Pro. In particular examples involving the substitution of charged, polar amino acids, if a first amino acid is one of His; Arg; Lys; Asp; and Glu, the first amino acid may be replaced by a second, different amino acid selected from His; Arg; Lys; Asp; and Glu. In further examples involving the substitution of charged, polar amino acids, if a first amino acid is one of Arg; Lys; Asp; and Glu, the first amino acid may be replaced by a second, different amino acid selected from Arg; Lys; Asp; and Glu. In particular examples involving the substitution of positively charged (basic), polar amino acids, if a first amino acid is one of His; Arg; and Lys, the first amino acid may be replaced by a second, different amino acid selected from His; Arg; and Lys. In further examples involving the substitution of positively charged, polar amino acids, if a first amino acid is Arg or Lys, the first amino acid may be replaced by the other amino acid of Arg and Lys. In particular examples involving the substitution of negatively charged (acidic), polar amino acids, if a first amino acid is Asp or Glu, the first amino acid may be replaced by the other amino acid of Asp and Glu.

In some embodiments, a conservative substitution includes the substitution of a first electrically neutral amino acid for a second, different electrically neutral amino acid. For example, if a first amino acid is one of Gly; Ser; Thr; Cys; Asn; Gln; and Tyr, the first amino acid may be replaced by a second, different amino acid selected from Gly; Ser; Thr; Cys; Asn; Gln; and Tyr.

In some embodiments, a conservative substitution includes the substitution of a first non-polar amino acid for a second, different non-polar amino acid. For example, if a first amino acid is one of Ala; Val; Leu; Be; Phe; Trp; Pro; and Met, the first amino acid may be replaced by a second, different amino acid selected from Ala; Val; Leu; Be; Phe; Trp; Pro; and Met.

In many examples, the selection of a particular second amino acid to be used in a conservative substitution to replace a first amino acid may be made in order to maximize the number of the foregoing classes to which the first and second amino acids both belong. Thus, if the first amino acid is Ser (a polar, non-aromatic, and electrically neutral amino acid), the second amino acid may be another polar amino acid (i.e., Thr; Asn; Gln; Cys; Gly; Pro; Arg; His; Lys; Asp; or Glu); another non-aromatic amino acid (i.e., Thr; Asn; Gln; Cys; Gly; Pro; Arg; His; Lys; Asp; Glu; Ala; Be; Leu; Val; or Met); or another electrically-neutral amino acid (i.e., Gly; Thr; Cys; Asn; Gln; or Tyr). However, it may be preferred that the second amino acid in this case be one of Thr; Asn; Gln; Cys; and Gly, because these amino acids share all the classifications according to polarity, non-aromaticity, and electrical neutrality. Additional criteria that may optionally be used to select a particular second amino acid to be used in a conservative substitution are known in the art. For example, when Thr; Asn; Gln; Cys; and Gly are available to be used in a conservative substitution for Ser, Cys may be eliminated from selection in order to avoid the formation of undesirable cross-linkages and/or disulfide bonds. Likewise, Gly may be eliminated from selection, because it lacks an alkyl side chain. In this case, Thr may be selected, e.g., in order to retain the functionality of a side chain hydroxyl group.

As used herein, the phrase “selectable marker” or “selectable marker gene” refers to a gene that is optionally used in plant transformation to, for example, protect the plant cells from a selective agent or provide resistance/tolerance to a selective agent. Only those cells or plants that receive a functional selectable marker are capable of dividing or growing under conditions having a selective agent. Examples of selective agents can include, for example, antibiotics, including spectinomycin, neomycin, kanamycin, paromomycin, gentamicin, and hygromycin. These selectable markers include gene for neomycin phosphotransferase (npt II), which expresses an enzyme conferring resistance to the antibiotic kanamycin, and genes for the related antibiotics neomycin, paromomycin, gentamicin, and G418, or the gene for hygromycin phosphotransferase (hpt), which expresses an enzyme conferring resistance to hygromycin. Other selectable marker genes can include genes encoding herbicide resistance including Bar (resistance against BASTA® (glufosinate ammonium), or phosphinothricin (PPT)), acetolactate synthase (ALS, resistance against inhibitors such as sulfonylureas (SUs), imidazolinones (IMIs), triazolopyrimidines (TPs), pyrimidinyl oxybenzoates (POBs), and sulfonylamino carbonyl triazolinones that prevent the first step in the synthesis of the branched-chain amino acids), glyphosate, 2,4-D, and metal resistance or sensitivity. The phrase “marker-positive” refers to plants that have been transformed to include the selectable marker gene.

Various selectable or detectable markers can be incorporated into the chosen expression vector to allow identification and selection of transformed plants, or transformants. Many methods are available to confirm the expression of selection markers in transformed plants, including for example DNA sequencing and PCR (polymerase chain reaction), Southern blotting, RNA blotting, immunological methods for detection of a protein expressed from the vector, e g., precipitated protein that mediates phosphinothricin resistance, or other proteins such as reporter genes β-glucuronidase (GUS), luciferase, green fluorescent protein (GFP), DsRed, β-galactosidase, chloramphenicol acetyltransferase (CAT), alkaline phosphatase, and the like (See Sambrook, et al., Molecular Cloning: A Laboratory Manual, Third Edition, Cold Spring Harbor Press, N.Y., 2001, the content of which is incorporated herein by reference in its entirety).

Selectable marker genes are utilized for the selection of transformed cells or tissues. Selectable marker genes include genes encoding antibiotic resistance, such as those encoding neomycin phosphotransferase II (NEO) and hygromycin phosphotransferase (HPT) as well as genes conferring resistance to herbicidal compounds. Herbicide resistance genes generally code for a modified target protein insensitive to the herbicide or for an enzyme that degrades or detoxifies the herbicide in the plant before it can act. For example, resistance to glyphosate or has been obtained by using genes coding for the mutant target enzymes, 5-enolpyruvylshikimate-3-phosphate synthase (EPSPS). Genes and mutants for EPSPS have been disclosed in U.S. Pat. Nos. 4,940,835, 5,188,642, 5,310,667, 5,633,435, 5,633,448, and 6,566,587, the contents of which are incorporated by reference in their entireties. Resistance to glufosinate ammonium, bromoxynil, and 2,4-dichlorophenoxyacetate (2,4-D) have been obtained by using bacterial genes encoding phosphinothricin acetyltransferase, a nitrilase, or a 2,4-dichlorophenoxyacetate monooxygenase, which detoxify the respective herbicides. Enzymes/genes for glufosinate resistance/tolerance have been disclosed in U.S. Pat. Nos. 5,273,894, 5,276,268, 5,550,318, and 5,561,236, the contents of which are incorporated by reference in their entireties. Enzymes/genes for 2,4-D resistance have been previously disclosed in U.S. Pat. Nos. 6,100,446 and 6,153,401, as well as patent applications US 2009/0093366 (AAD-1) and WO 2007/053482 (AAD-12), the contents of which are hereby incorporated by reference in their entireties. Enzymes/genes for nitrilase have been previously disclosed in U.S. Pat. No. 4,810,648, the content of which is incorporated by reference in its entirety.

Other herbicides can inhibit the growing point or meristem, including imidazolinone or sulfonylurea, and genes for resistance/tolerance of acetohydroxyacid synthase (AHAS) and acetolactate synthase (ALS) for these herbicides have been described. Genes and mutants for AHAS and mutants have been disclosed in U.S. Pat. Nos. 4,761,373, 5,304,732, 5,331,107, 5,853,973, and 5,928,937, the contents of which are incorporated by reference in their entireties. Genes and mutants for ALS have been disclosed in U.S. Pat. Nos. 5,013,659 and 5,141,870, the contents of which are incorporated by reference in their entireties.

Glyphosate resistance genes include mutant 5-enolpyruvylshikimate-3-phosphate synthase (EPSPs) genes (via the introduction of recombinant nucleic acids and/or various forms of in vivo mutagenesis of native EPSPs genes), aroA genes and glyphosate acetyl transferase (GAT) genes, respectively). Resistance genes for other phosphono compounds include glufosinate (phosphinothricin acetyl transferase (PAT) genes from Streptomyces species, including Streptomyces hygroscopicus and Streptomyces viridichromogenes), and pyridinoxy or phenoxy proprionic acids and cyclohexones (ACCase inhibitor-encoding genes). Herbicide resistance/tolerance genes of acetyl coemzyme A carboxylase (ACCase) have been described in U.S. Pat. Nos. 5,162,602 and 5,498,544, the contents of which are incorporated by reference in their entireties.

A DNA molecule encoding a mutant aroA gene can be obtained under ATCC accession number 39256, and the nucleotide sequence of the mutant gene is disclosed in U.S. Pat. No. 4,769,061 to Comai, European patent application No. 0 333 033 to Kumada et al., and U.S. Pat. No. 4,975,374 to Goodman et al., disclosing nucleotide sequences of glutamine synthetase genes which confer resistance to herbicides such as L-phosphinothricin. The nucleotide sequence of a PAT gene is provided in European application No. 0 242 246 to Leemans et al. Also DeGreef et al., Bio/Technology 7:61 (1989), describes the production of transgenic plants that express chimeric bar genes coding for PAT activity. Exemplary of genes conferring resistance to phenoxy proprionic acids and cyclohexones, including sethoxydim and haloxyfop, are the Acc1-S1, Acc1-S2 and Acc1-S3 genes described by Marshall et al., Theon. Appl. Genet. 83:435 (1992). GAT genes capable of conferring glyphosate resistance are described in WO 2005012515 to Castle et al. Genes conferring resistance to 2,4-D, fop and pyridyloxy auxin herbicides are described in WO 2005107437 and U.S. patent application Ser. No. 11/587,893.

Other herbicides can inhibit photosynthesis, including triazine (psbA and 1s+ genes) or benzonitrile (nitrilase gene). Przibila et al., Plant Cell 3:169 (1991), describes the transformation of Chlamydomonas with plasmids encoding mutant psbA genes. Nucleotide sequences for nitrilase genes are disclosed in U.S. Pat. No. 4,810,648 to Stalker, and DNA molecules containing these genes are available under ATCC Accession Nos. 53435, 67441, and 67442. Cloning and expression of DNA coding for a glutathione S-transferase is described by Hayes et al., Biochem. J. 285:173 (1992).

For purposes of the present invention, selectable marker genes include, but are not limited to genes encoding: neomycin phosphotransferase II (Fraley et al. (1986) CRC Critical Reviews in Plant Science, 4:1-25); cyanamide hydratase (Maier-Greiner et al. (1991) Proc. Natl. Acad. Sci. USA, 88:4250-4264); aspartate kinase; dihydrodipicolinate synthase (Perl et al. (1993) Bio/Technology, 11:715-718); tryptophan decarboxylase (Goddijn et al. (1993) Plant Mol. Bio., 22:907-912); dihydrodipicolinate synthase and desensitized aspartade kinase (Perl et al. (1993) Bio/Technology, 11:715-718); bar gene (Toki et al. (1992) Plant Physiol., 100:1503-1507 and Meagher et al. (1996) and Crop Sci., 36:1367); tryptophan decarboxylase (Goddijn et al. (1993) Plant Mol. Biol., 22:907-912); neomycin phosphotransferase (NEO) (Southern et al. (1982) J. Mol. Appl. Gen., 1:327; hygromycin phosphotransferase (HPT or HYG) (Shimizu et al. (1986) Mol. Cell Biol., 6:1074); dihydrofolate reductase (DHFR) (Kwok et al. (1986) PNAS USA 4552); phosphinothricin acetyltransferase (DeBlock et al. (1987) EMBO J., 6:2513); 2,2-dichloropropionic acid dehalogenase (Buchanan-Wollatron et al. (1989) J. Cell. Biochem. 13D:330); acetohydroxyacid synthase (Anderson et al., U.S. Pat. No. 4,761,373; Haughn et al. (1988) Mol. Gen. Genet. 221:266); 5-enolpyruvyl-shikimate-phosphate synthase (aroA) (Comai et al. (1985) Nature 317:741); haloarylnitrilase (Stalker et al., published PCT application WO87/04181); acetyl-coenzyme A carboxylase (Parker et al. (1990) Plant Physiol. 92:1220); dihydropteroate synthase (sul I) (Guerineau et al. (1990) Plant Mol. Biol. 15:127); and 32 kD photosystem II polypeptide (psbA) (Hirschberg et al. (1983) Science, 222:1346).

Also included are genes encoding resistance to: chloramphenicol (Herrera-Estrella et al. (1983) EMBO J., 2:987-992); methotrexate (Herrera-Estrella et al. (1983) Nature, 303:209-213; Meijer et al. (1991) Plant Mol Bio., 16:807-820 (1991); hygromycin (Waldron et al. (1985) Plant Mol. Biol., 5:103-108; Zhijian et al. (1995) Plant Science, 108:219-227 and Meijer et al. (1991) Plant Mol. Bio. 16:807-820); streptomycin (Jones et al. (1987) Mol. Gen. Genet., 210:86-91); spectinomycin (Bretagne-Sagnard et al. (1996) Transgenic Res., 5:131-137); bleomycin (Hille et al. (1986) Plant Mol. Biol., 7:171-176); sulfonamide (Guerineau et al. (1990) Plant Mol. Bio., 15:127-136); bromoxynil (Stalker et al. (1988) Science, 242:419-423); 2,4-D (Streber et al. (1989) Bio/Technology, 7:811-816); glyphosate (Shaw et al. (1986) Science, 233:478-481); and phosphinothricin (DeBlock et al. (1987) EMBO J., 6:2513-2518). All references recited in the disclosure are hereby incorporated by reference in their entireties unless stated otherwise.

The above list of selectable marker and reporter genes are not meant to be limiting. Any reporter or selectable marker gene are encompassed by the present invention. If necessary, such genes can be sequenced by methods known in the art.

The reporter and selectable marker genes are synthesized for optimal expression in the plant. That is, the coding sequence of the gene has been modified to enhance expression in plants. The synthetic marker gene is designed to be expressed in plants at a higher level resulting in higher transformation efficiency. Methods for synthetic optimization of genes are available in the art. In fact, several genes have been optimized to increase expression of the gene product in plants.

The marker gene sequence can be optimized for expression in a particular plant species or alternatively can be modified for optimal expression in plant families. The plant preferred codons may be determined from the codons of highest frequency in the proteins expressed in the largest amount in the particular plant species of interest. See, for example, EPA 0359472; EPA 0385962; WO 91/16432; Perlak et al. (1991) Proc. Natl. Acad. Sci. USA, 88:3324-3328; and Murray et al. (1989) Nucleic Acids Research, 17: 477-498; U.S. Pat. No. 5,380,831; and U.S. Pat. No. 5,436,391, herein incorporated by reference. In this manner, the nucleotide sequences can be optimized for expression in any plant. It is recognized that all or any part of the gene sequence may be optimized or synthetic. That is, fully optimized or partially optimized sequences may also be used.

In addition, several transformation strategies utilizing the Agrobacterium-mediated transformation system have been developed. For example, the binary vector strategy is based on a two-plasmid system where T-DNA is in a different plasmid from the rest of the Ti plasmid. In a co-integration strategy, a small portion of the T-DNA is placed in the same vector as the foreign gene, which vector subsequently recombines with the Ti plasmid.

As used herein, the phrase “plant” includes dicotyledons plants and monocotyledons plants. Examples of dicotyledons plants include tobacco, Arabidopsis, soybean, tomato, papaya, canola, sunflower, cotton, alfalfa, potato, grapevine, pigeon pea, pea, Brassica, chickpea, sugar beet, rapeseed, watermelon, melon, pepper, peanut, pumpkin, radish, spinach, squash, broccoli, cabbage, carrot, cauliflower, celery, Chinese cabbage, cucumber, eggplant, and lettuce. Examples of monocotyledons plants include corn, rice, wheat, sugarcane, barley, rye, sorghum, orchids, bamboo, banana, cattails, lilies, oat, onion, millet, and triticale.

As used herein, the phrase “plant cell described” or “transformed plant cell” refers to a plant cell that is transformed with stably-integrated, non-natural, recombinant DNA, e.g., by Agrobacterium-mediated transformation or by bombardment using microparticles coated with recombinant DNA or other means. A plant cell of this invention can be an originally-transformed plant cell that exists as a microorganism or as a progeny plant cell that is regenerated into differentiated tissue, e.g., into a transgenic plant with stably-integrated, non-natural recombinant DNA, or seed or pollen derived from a progeny transgenic plant.

As used herein, the phrase “consensus sequence” refers to an artificial sequence of amino acids in a conserved region of an alignment of amino acid sequences of homologous proteins, e.g., as determined by a CLUSTALW alignment of amino acid sequence of (functional) homolog proteins.

As used herein, the phrase “yield” or “plant yield” refers to increased plant growth, increased crop growth, increased biomass, and/or increased plant product production (including grain), and is dependent to some extent on temperature, plant size, organ size, planting density, light, water and nutrient availability, and how the plant copes with various stresses, including through temperature acclimation and water or nutrient use efficiency.

Increased yield of a transgenic plant of the present invention can be measured in a number of ways, including plant volume, plant biomass, test weight, seed number per plant, seed weight, seed number per unit area (i.e., seeds, or weight of seeds, per acre), bushels per acre (bu/a), tonnes per acre, tons per acre, and/or kilo per hectare. For trees, yield could be measured as average wood production per year over the rotation cycle. For example, fresh weight yield may be determined for plants or plant parts at the end of the vegetative phase of a crop before drying. Dry weight yield may be similarly determined after a period of water removal. Both fresh and dry weight yield may be determined with a balance.

As used herein, the phrase “homolog” refers to a nucleic acid or a protein in a group of proteins that perform the same biological function, e.g., proteins that belong to the same protein family or similar nucleic acids that provide a common enhanced trait in transgenic plants of this invention. Homologs are expressed by homologous genes. With reference to homologous genes, homologs include orthologs, i.e., genes expressed in different species that evolved from common ancestral genes by speciation and encode proteins that retain the same function, but do not include paralogs, i.e., genes that are related by duplication but have evolved to encode proteins with different functions. Homologous genes include naturally occurring alleles and artificially-created variants. Degeneracy of the genetic code provides the possibility to substitute at least one base of the protein encoding sequence of a gene with a different base without causing the amino acid sequence of the polypeptide produced from the gene to be changed. When optimally aligned, homolog genes have at least 50%, 60%, 70%, 80%, 85%, 90%, 95%, 96%, 97%, 98% or 99% or more sequence identity over the full length of the gene identified as being associated with imparting an enhanced trait when expressed in plant cells. In one aspect of the invention homolog genes have a nucleic acid or amino acid sequence similarity that has at least 85%, 90%, 95%, 96%, 97%, 98% or 99% identity to a consensus sequence of proteins, nucleotides and homologs disclosed herein.

Homologs can be identified by comparison of amino acid sequence, e.g., manually or by use of a computer-based tool using known homology-based search algorithms such as those commonly known and referred to as BLAST, FASTA, and Smith-Waterman. A local sequence alignment program, (e.g., Basic Local Alignment Search Tool (BLAST)) can be used to search a database of sequences to find similar sequences, and the summary Expectation value (E-value) used to measure the sequence base similarity. Because a protein hit with the best E-value for a particular organism may not necessarily be an ortholog, i.e., have the same function, or be the only ortholog, a reciprocal query is used to filter hit sequences with significant E-values for ortholog identification. The reciprocal query entails search of the significant hits against a database of amino acid sequences from the base organism that are similar to the sequence of the query protein. A hit can be identified as an ortholog, when the reciprocal query's best bit is the query protein itself or a protein encoded by a duplicated gene after speciation. A further aspect of the homologs encoded by DNA useful in the transgenic plants of the invention are those proteins that differ from a disclosed protein as the result of deletion or insertion of one or more amino acids in a native sequence.

As used herein, the phrase “percent identity” or “% identity” refers to the extent to which the sequences of DNA or protein segments are invariant throughout a window of alignment of sequences, for example nucleotide sequences or amino acid sequences. An “identity fraction” for a sequence aligned with a reference sequence is the number of identical components which are shared by the sequences, divided by the length of the alignment not including gaps introduced by the alignment algorithm. “Percent identity” is the identity fraction times 100. Percent identity is calculated over the aligned length preferably using a local alignment algorithm, for example BLASTp.

The term “sequence identity” or “identity,” as used herein in the context of two nucleic acid or polypeptide sequences, may refer to residues in two sequences that are the same when aligned for maximum correspondence over a specified comparison window.

As used herein, the term “percentage of sequence identity” may refer to the value determined by comparing two optimally aligned sequences (e.g., nucleic acid sequences, and amino acid sequences) over a comparison window, wherein the portion of the sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleotide or amino acid residue occurs in both sequences to yield the number of matched positions, dividing the number of matched positions by the total number of positions in the comparison window, and multiplying the result by 100 to yield the percentage of sequence identity.

As used herein, the term “substantially identical” may refer to nucleotide sequences that are more than 85% identical. For example, a substantially identical nucleotide sequence may be at least 85.5%; at least 86%; at least 87%; at least 88%; at least 89%; at least 90%; at least 91%; at least 92%; at least 93%; at least 94%; at least 95%; at least 96%; at least 97%; at least 98%; at least 99%; or at least 99.5% identical to a reference sequence.

Specifically hybridizable/Specifically complementary: A first nucleic acid molecule (e.g., a “probe”) may be “specifically hybridizable” or “specifically complementary” to a second nucleic acid (“DNA target”). “Specifically hybridizable” and “specifically complementary” are terms that indicate a sufficient degree of complementarity such that stable and specific binding occurs between the first nucleic acid molecule and the DNA target. A nucleic acid molecule need not be 100% complementary to its target sequence to be specifically hybridizable. A nucleic acid molecule is specifically hybridizable when there is a sufficient degree of complementarity to avoid non-specific binding of the nucleic acid to non-target sequences under conditions where specific binding is desired, for example, under stringent hybridization conditions.

Hybridization conditions resulting in particular degrees of stringency will vary depending upon the nature of the hybridization method of choice and the composition and length of the hybridizing nucleic acid sequences. Generally, the temperature of hybridization and the ionic strength (especially the Na⁺ and/or Mg⁺⁺ concentration) of the hybridization buffer will determine the stringency of hybridization, though wash times also influence stringency. Calculations regarding hybridization conditions required for attaining particular degrees of stringency are known to those of ordinary skill in the art, and are discussed, for example, in Sambrook et al. (ed.) Molecular Cloning: A Laboratory Manual, 2^{nd} ed., vol. 1-3, Cold Spring Harbor Laboratory Press, Cold Spring Harbor, N.Y., 1989, chapters 9 and 11; and Hames and Higgins (eds.) Nucleic Acid Hybridization, IRL Press, Oxford, 1985. Further detailed instruction and guidance with regard to the hybridization of nucleic acids may be found, for example, in Tijssen, “Overview of principles of hybridization and the strategy of nucleic acid probe assays,” in Laboratory Techniques in Biochemistry and Molecular Biology—Hybridization with Nucleic Acid Probes, Part I, Chapter 2, Elsevier, N Y, 1993; and Ausubel et al., Eds., Current Protocols in Molecular Biology, Chapter 2, Greene Publishing and Wiley-Interscience, N Y, 1995.

As used herein, “stringent conditions” encompass conditions under which hybridization will only occur if there is less than 25% mismatch between the hybridization molecule and the DNA target. “Stringent conditions” include further particular levels of stringency. Thus, as used herein, “moderate stringency” conditions are those under which molecules with more than 25% sequence mismatch will not hybridize; conditions of “medium stringency” are those under which molecules with more than 15% mismatch will not hybridize; and conditions of “high stringency” are those under which sequences with more than 10% mismatch will not hybridize. Conditions of “very high stringency” are those under which sequences with more than 6% mismatch will not hybridize.

In particular embodiments, stringent conditions are hybridization at 65° C. in 6× saline-sodium citrate (SSC) buffer, 5×Denhardt's solution, 0.5% SDS, and 100 μg sheared salmon testes DNA, followed by 15-30 minute sequential washes at 65° C. in 2×SSC buffer and 0.5% SDS, followed by 1×SSC buffer and 0.5% SDS, and finally 0.2×SSC buffer and 0.5% SDS.

As used herein, two nucleic acid sequence molecules are said to exhibit “complete complementarity” when every nucleotide of a sequence read in the 5′ to 3′ direction is complementary to every nucleotide of the other sequence when read in the 3′ to 5′ direction. A nucleotide sequence that is complementary to a reference nucleotide sequence will exhibit a sequence identical to the reverse complement sequence of the reference nucleotide sequence. These terms and descriptions are well-defined in the art and are easily understood by those of ordinary skill in the art.

As used herein, the phrase “functional activity” or “functionally active” refers to the proteins/enzymes for use according to the subject invention which have the ability to provide for stress tolerance which can result in an increased yield. Transfer of the functional activity to plant or bacterial systems can involve a nucleic acid sequence, encoding the amino acid sequence for a protein of the subject invention, integrated into a protein expression vector appropriate to the host in which the vector will reside. One way to obtain a nucleic acid sequence encoding a protein with functional activity is to isolate the native genetic material from the bacterial species which produces the protein of interest, using information deduced from the protein's amino acid sequence, as disclosed herein. The native sequences can be optimized for expression in plants. Optimized polynucleotides can also be designed based on the protein sequence.

As used herein, the phrase “gene fusion construct” refers to a recombinant nucleic acid sequence comprising cojoined sequences derived from at least two different parental nucleic acids or a chimeric DNA. A “modified gene fusion construct” comprises a subset of gene fusion constructs, in which at least one nucleotide (optionally, in a coding region or linker region) in the construct is modified, or changed, as compared to a parent or wild-type sequence from which that portion of the construct is derived.

As used herein, the phrases “control sequences” and “regulatory sequences” are interchangeable and refer to nucleic acid sequences useful for transcription/gene expression in plants. “Control sequences” or “regulatory sequences” may include, but not limited to, promoters, operators, enhancers, origins of replication, ribosome binding sites, termination and polyadenylation signals.

As used herein, the phrase “promoter” refers to regulatory DNA for initializing transcription. A “plant promoter” is a promoter capable of initiating transcription in plant cells whether or not its origin is a plant cell, e.g., is it well known that Agrobacterium T-DNA promoters are functional in plant cells. Thus, plant promoters include promoter DNA obtained from plants, plant viruses and bacteria such as Agrobacterium and Bradyrhizobium bacteria. Examples of promoters under developmental control include promoters that preferentially initiate transcription in certain tissues, including leaves, roots, or seeds. Such promoters are referred to as “tissue preferred.” Promoters that initiate transcription only in certain tissues are referred to as “tissue specific.” A “cell type” specific promoter primarily drives expression in certain cell types in one or more organs, for example, vascular cells in roots or leaves. An “inducible” or “repressible” promoter is a promoter which is under environmental or chemical control. Examples of environmental conditions that may affect transcription by inducible promoters include anaerobic conditions, or certain chemicals, or the presence of light. Tissue specific, tissue preferred, cell type specific, and inducible promoters belong to the class of “non-constitutive” promoters. A “constitutive” promoter refers to a promoter which is active under most conditions and in most tissues.

Numerous promoters that are active in plant cells have been previously described. These include promoters present in plant genomes as well as promoters from other sources, including nopaline synthase (NOS) promoter and octopine synthase (OCS) promoters carried on tumor-inducing plasmids of Agrobacterium tumefaciens and the CaMV35S promoters from the cauliflower mosaic virus as disclosed in U.S. Pat. Nos. 5,164,316 and 5,322,938. Useful promoters derived from plant genes are found in U.S. Pat. No. 5,641,876 disclosing a rice actin promoter; U.S. Pat. No. 7,151,204 disclosing a maize chloroplast aldolase promoter and a maize aldolase (FDA) promoter; and U.S. Patent Application Publication 2003/0131377 disclosing a maize nicotianamine synthase promoter. These and numerous other promoters that function in plant cells are known to those skilled in the art and available for use in recombinant polynucleotides described herein to provide for expression of desired genes in transgenic plant cells.

Furthermore, the promoters may be altered to contain multiple “enhancer sequences” to assist in elevating gene expression. Such enhancers are known in the art. By including an enhancer sequence with such constructs, the expression of the selected protein may be enhanced. These enhancers often are found 5′ to the start of transcription in a promoter that functions in eukaryotic cells, but can often be inserted upstream (5′) or downstream (3′) to the coding sequence. In some instances, these 5′ enhancing elements are introns. Particularly useful as enhancers are the 5′ introns of the rice actin 1 (see for example U.S. Pat. No. 5,641,876) and rice actin 2 genes, the maize alcohol dehydrogenase gene intron(s), the maize heat shock protein 70 gene intron (see for example U.S. Pat. No. 5,593,874) and the maize shrunken 1 gene intron. See also U.S. Patent Application Publication 2002/0192813A1 disclosing 5′, 3′ and intron elements useful in the design of effective plant expression vectors.

In some embodiments, sufficient expression in plant seed tissues is desired to affect improvements in seed composition. Exemplary promoters for use for seed composition modification include promoters from seed genes such as napin as disclosed in U.S. Pat. No. 5,420,034, maize L3 oleosin as disclosed in U.S. Pat. No. 6,433,252), zein Z27 as disclosed by Russell et al. (1997) Transgenic Res. 6(2): 157-166), globulin 1 as disclosed by Belanger et al (1991) Genetics 129:863-872), glutelin 1 as disclosed by Russell (1997, supra), and peroxiredoxin antioxidant (Perl) as disclosed by Stacy et al. (1996) Plant Mol Biol. 31(6):1205-1216.

As used herein, the phrase “operably linked” refers to the association of two or more DNA fragments in a recombinant DNA construct so that the function of one, e.g., protein-encoding DNA, is controlled by the other, e.g., a promoter. A first nucleotide sequence is “operably linked” with a second nucleotide sequence when the first nucleotide sequence is in a functional relationship with the second nucleotide sequence. For instance, a promoter is operably linked to a coding sequence if the promoter affects the transcription or expression of the coding sequence. When recombinantly produced, operably linked nucleotide sequences are generally contiguous and, where necessary to join two protein-coding regions, in the same reading frame. However, nucleotide sequences need not be contiguous to be operably linked.

Recombinant DNA constructs are assembled using methods well known to persons of ordinary skill in the art and typically comprise a promoter operably linked to DNA, the expression of which provides the enhanced agronomic trait. Other construct components may include additional regulatory elements, including 5′ leaders and introns for enhancing transcription, 3′ untranslated regions (for example, polyadenylation signals and sites), and DNA for transit or signal peptides.

Recombinant DNA constructs described herein also generally include a 3′ element that typically contains a polyadenylation signal and site. Well-known 3′ elements include those from Agrobacterium tumefaciens genes such as nos 3′, tml 3′, tmr 3′, tms 3′, ocs 3′, tr73′, for example disclosed in U.S. Pat. No. 6,090,627; 3′ elements from plant genes such as wheat (Triticum aesevitum) heat shock protein 17 (Hspl 73), a wheat ubiquitin gene, a wheat fructose-1,6-biphosphatase gene, a rice glutelin gene, a rice lactate dehydrogenase gene and a rice beta-tubulin gene, all of which are disclosed in U.S. Patent Application Publication 2002/0192813; and the pea (Pisum sativum) ribulose biphosphate carboxylase gene (rbs 3′), and 3′ elements from the genes within the host plant.

Constructs and vectors may also include a transit peptide for targeting of a gene to a plant organelle, particularly to a chloroplast, leucoplast or other plastid organelle. For descriptions of the use of chloroplast transit peptides see for example U.S. Pat. No. 5,188,642 and U.S. Pat. No. 5,728,925.

As used herein, the phrase “expressed” refers to a protein which is expressed or produced in a plant cell when its cognate DNA is transcribed to mRNA that is translated to the protein. As used herein, the phrase “suppressed” refers to decreased expression or activity of a protein. Typically a protein is suppressed in a plant cell when there is a decrease in the amount and/or activity of the protein in the plant cell. The presence or activity of the protein can be decreased by any amount up to and including a total loss of protein expression and/or activity.

Recombinant DNA constructs for gene suppression can be designed for any of a number the well-known methods for suppressing transcription of a gene, the accumulation of the mRNA corresponding to that gene or preventing translation of the transcript into protein. Posttranscriptional gene suppression can be practically effected by transcription of RNA that forms double-stranded RNA (dsRNA) having homology to mRNA produced from a gene targeted for suppression.

Gene suppression can also be achieved by insertion mutations created by transposable elements that prevent gene function. For example, in many dicot plants, transformation with the T-DNA of Agrobacterium may be readily achieved and large numbers of transformants can be rapidly obtained. Also, some species have lines with active transposable elements that can efficiently be used for the generation of large numbers of insertion mutations, while some other species lack such options. Mutant plants produced by Agrobacterium or transposon mutagenesis and having altered expression of a polypeptide of interest can be identified using the polynucleotides of the present invention. For example, a large population of mutated plants may be screened with polynucleotides encoding the polypeptide of interest to detect mutated plants having an insertion in the gene encoding the polypeptide of interest.

As used herein, the phrase “control plant” refers to a plant that does not contain the recombinant DNA that imparts an enhanced trait. A control plant is used to identify and select a transgenic plant that has an enhanced trait. A suitable control plant can be a non-transgenic plant of the parental line used to generate a transgenic plant, i.e., devoid of recombinant DNA. A suitable control plant may in some cases be a progeny of a hemizygous transgenic plant line that does not contain the recombinant DNA.

As used herein, the phrase “enhanced trait” refers to a characteristic of a transgenic plant that includes, but is not limited to, an enhanced agronomic trait characterized by enhanced plant morphology, physiology, growth and development, yield, nutritional enhancement, disease or pest resistance, or environmental or chemical tolerance. In some embodiments, enhanced trait is selected from a group of enhanced traits consisting of enhanced water use efficiency, enhanced cold tolerance, increased yield, enhanced nitrogen use efficiency, enhanced seed protein and enhanced seed oil. In some embodiments, the enhanced trait is enhanced yield including increased yield under non-stress conditions and increased yield under environmental stress conditions. Stress conditions may include at least one of, for example, drought, shade, fungal disease, viral disease, bacterial disease, insect infestation, nematode infestation, cold temperature exposure, heat exposure, osmotic stress, reduced nitrogen nutrient availability, reduced phosphorus nutrient availability and high plant density.

“Yield” can be affected by many properties including without limitation, plant height, pod number, pod position on the plant, number of internodes, incidence of pod shatter, grain size, grain number per pod or ear or spikelet/plant, efficiency of nodulation and nitrogen fixation, efficiency of nutrient assimilation, resistance to biotic and abiotic stress, carbon assimilation, plant architecture, resistance to lodging, percent seed germination, seedling vigor, and juvenile traits. Yield can also be affected by efficiency of germination (including germination in stressed conditions), growth rate (including growth rate in stressed conditions), ear number, seed number per ear, seed size, composition of seed (starch, oil, protein) and characteristics of seed fill.

Increased yield of a transgenic plant described herein can be measured in a number of ways, including test weight, seed number per plant, seed weight, seed number per unit area (i.e., seeds, or weight of seeds, per acre), bushels per acre, tons per acre, or kilo per hectare. For example, corn yield may be measured as production of shelled corn kernels per unit of production area, for example in bushels per acre or metric tons per hectare, often reported on a moisture adjusted basis. Increased yield may result from improved utilization of key biochemical compounds, including nitrogen, phosphorous and carbohydrate, or from improved responses to environmental stresses, including cold, heat, drought, salt, and attack by pests or pathogens. Recombinant DNA described herein can also be used to provide plants having improved growth and development, and ultimately increased yield, as the result of modified expression of plant growth regulators or modification of cell cycle or photosynthesis pathways. Also provided is the generation of transgenic plants that demonstrate enhanced yield with respect to a seed component that may or may not correspond to an increase in overall plant yield. Such properties include enhancements in seed oil, seed molecules such as protein and starch, oil components as may be manifested by an alteration in the ratios of seed components.

Transgenic plants may comprise a stack of one or more polynucleotides disclosed herein resulting in the production or suppression of multiple polypeptide sequences. Transgenic plants comprising stacks of polynucleotide sequences can be obtained by either or both of traditional breeding methods or through genetic engineering methods. These methods include, but are not limited to, breeding individual lines each comprising a polynucleotide of interest, transforming a transgenic plant comprising a gene disclosed herein with a subsequent gene, and co-transformation of genes into a single plant cell. Co-transformation of genes can be carried out using single transformation vectors comprising multiple genes or genes carried separately on multiple vectors.

Transgenic plants comprising or derived from plant cells of this invention transformed with recombinant DNA can be further enhanced with stacked traits, e.g., a crop plant having an enhanced trait resulting from expression of DNA disclosed herein in combination with herbicide and/or pest resistance traits. For example, genes of the current invention can be stacked with other traits of agronomic interest, including a trait providing herbicide resistance, or insect resistance, such as using a gene from Bacillus thuringiensis to provide resistance against lepidopteran, coleopteran, homopteran, hemipteran, and other insects.

Plant Cell Transformation Methods: Numerous methods for transforming chromosomes in a plant cell nucleus with recombinant DNA are known in the art and are used in methods of preparing a transgenic plant cell nucleus, and plant. Two effective methods for such transformation are Agrobacterium-mediated transformation and microprojectile bombardment. Microprojectile bombardment methods are described in U.S. Pat. No. 5,015,580 (soybean); U.S. Pat. No. 5,550,318 (corn); U.S. Pat. No. 5,538,880 (corn); U.S. Pat. No. 5,914,451 (soybean); U.S. Pat. No. 6,160,208 (corn); U.S. Pat. No. 6,399,861 (corn); U.S. Pat. No. 6,153,812 (wheat) and U.S. Pat. No. 6,365,807 (rice). Agrobacterium-mediated transformations have been described in U.S. Pat. No. 5,159,135 (cotton); U.S. Pat. No. 5,824,877 (soybean); U.S. Pat. No. 5,463,174 (canola); U.S. Pat. No. 5,591,616 (corn); U.S. Pat. No. 5,846,797 (cotton); U.S. Pat. No. 6,384,301 (soybean), U.S. Pat. No. 7,026,528 (wheat) and U.S. Pat. No. 6,329,571 (rice), U.S. Patent Application Publication 2004/0087030 (cotton), and U.S. Patent Application Publication 2001/0042257 (sugar beet), all of which are incorporated herein by reference for enabling the production of transgenic plants.

Transformation of plant material is typically practiced in tissue culture on a nutrient media, i.e., a mixture of nutrients that will allow cells to grow in vitro. Recipient cell targets include, but are not limited to, meristem cells, hypocotyls, calli, immature embryos and gametic cells such as microspores, pollen, sperm and egg cells. Callus may be initiated from tissue sources including, but not limited to, immature embryos, hypocotyls, seedling apical meristems, microspores and the like. Cells containing a transgenic nucleus are grown into transgenic plants.

In addition to direct transformation of a plant material with a recombinant DNA, a transgenic plant cell nucleus can be prepared by crossing a first plant having cells with a transgenic nucleus with recombinant DNA with a second plant lacking the transgenic nucleus. A transgenic plant with recombinant DNA providing an enhanced trait, e.g., enhanced yield, can be crossed with a transgenic plant line having other recombinant DNA that confers another trait, for example herbicide resistance or pest resistance, to produce progeny plants having recombinant DNA that confers both traits. Typically, in such breeding for combining traits the transgenic plant donating the additional trait is a male line and the transgenic plant carrying the base traits is the female line. The progeny of this cross will segregate such that some of the plants will carry the DNA for both parental traits and some will carry DNA for one parental trait; such plants can be identified by markers associated with parental recombinant DNA, e.g., marker identification by analysis for recombinant DNA. In the case where a selectable marker is linked to the recombinant DNA, can also be identified by application of the selecting agent for example a herbicide for use with a herbicide tolerance marker, or by selection for the enhanced trait. Progeny plants carrying DNA for both parental traits can be crossed back into the female parent line multiple times, for example usually 6 to 8 generations, to produce a progeny plant with substantially the same genotype as one original transgenic parental line but for the recombinant DNA of the other transgenic parental line.

In the practice of transformation DNA is typically introduced into only a small percentage of target plant cells in any one transformation experiment. Marker genes are used to provide an efficient system for identification of those cells that are stably transformed by receiving and integrating a recombinant DNA molecule into their genomes. Preferred marker genes provide selective markers which confer resistance to a selective agent, such as an antibiotic or a herbicide as described above.

Plant cells that survive exposure to the selective agent, or plant cells that have been scored positive in a screening assay, may be cultured in regeneration media and allowed to mature into plants. Developing plantlets regenerated from transformed plant cells can be transferred to plant growth mix, and hardened off, for example, in an environmentally controlled chamber at about 85% relative humidity, 600 ppm CO₂, and 25-250 microEinsteins m^{′2} s^{′1} of light, prior to transfer to a greenhouse or growth chamber for maturation. Plants are regenerated from about 6 weeks to 10 months after a transformant is identified, depending on the initial tissue, and plant species. Plants may be pollinated using conventional plant breeding methods known to those of skill in the art and seed produced, for example self-pollination is commonly used with transgenic corn. The regenerated transformed plant or its progeny seed or plants can be tested for expression of the recombinant DNA and selected for the presence of enhanced agronomic trait.

Transgenic Plants and Seeds: Transgenic plants derived from transgenic plant cells having a transgenic nucleus provided herein are grown to generate transgenic plants having an enhanced trait as compared to a control plant and produce transgenic seed and haploid pollen of this invention. Such plants with enhanced traits can be identified by selection of transformed plants or progeny seed for the enhanced trait. A selection method is designed for efficiency to evaluate multiple transgenic plants/events comprising the recombinant DNA (for example, 5-10 plants each from 2 to 20 transgenic events). Transgenic plants grown from transgenic seeds provided herein demonstrate improved agronomic traits that contribute to increased yield or other trait that provides increased plant value, including, for example, improved seed quality. Improved traits provided herein include enhanced water use efficiency, enhanced cold tolerance, increased yield, enhanced nitrogen use efficiency, enhanced seed protein and enhanced seed oil.

The ability to control multiple pest problems through various traits is a valuable commercial product concept, and the convenience of this product concept is enhanced if insect control traits and/or weed control traits and/or agronomic traits are combined in the same plant. Further, improved value may be obtained via single plant combinations of Insect Resistance (IR) traits conferred by a Bacillus thuringiensis (B.t.) insecticidal protein with one or more additional Herbicide Tolerance (HT) traits such as those mentioned above, plus one or more additional input traits (e.g. other insect resistance conferred by B.t.-derived or other insecticidal proteins, insect resistance conferred by mechanisms such as RNAi and the like, disease resistance, stress tolerance, improved nitrogen utilization, and the like), or output traits (e.g., high oils content, healthy oil composition, nutritional improvement, and the like). Such combinations may be obtained either through conventional breeding (e.g., breeding stack) or jointly as a novel transformation event involving the simultaneous introduction of multiple genes (e.g., molecular stack). Such stacking may be performed using RNAi technology or through the use of EXZACT®. Co-transformation of genes can be carried out using single transformation vectors comprising multiple genes or genes carried separately on multiple vectors. Benefits include the ability to manage insect pests and improved weed control in a crop plant that provides secondary benefits to the producer and/or the consumer. Thus, the subject invention can be used to provide transformed plants with combinations of traits that comprise a complete agronomic package of improved crop quality with the ability to flexibly and cost effectively control any number of agronomic issues.

Genes that Confer Resistance to an Herbicide

A. Resistance/tolerance of acetohydroxyacid synthase (AHAS) and acetolactate synthase (ALS) against herbicides imidazolinone or sulfonylurea. Genes and mutants for AHAS and mutants have been disclosed in U.S. Pat. Nos. 4,761,373, 5,304,732, 5,331,107, 5,853,973, and 5,928,937. Genes and mutants for ALS have been disclosed in U.S. Pat. Nos. 5,013,659 and 5,141,870.

B. Resistance/tolerance genes of acetyl coemzyme A carboxylase (ACCase) against herbicides cyclohexanediones and/or aryloxyphenoxypropanoic acid (including Haloxyfop, Diclofop, Fenoxyprop, Fluazifop, Quizalofop) have been described in U.S. Pat. Nos. 5,162,602 and 5,498,544.

C. Genes for glyphosate resistance/tolerance. Gene of 5-enolpyruvyl-3-phosphoshikimate synthase (ES3P synthase) has been described in U.S. Pat. No. 4,769,601. Genes of 5-enolpyruvylshikimate-3-phosphate synthase (EPSPS) and mutants have been described in U.S. Pat. Nos. 4,940,835, 5,188,642, 5,310,667, 5,633,435, 5,633,448, and 6,566,587.

D. Genes for glufosinate (bialaphos, phosphinothricin (PPT)) resistance/tolerance. Gene for phosphinothricin acetyltransferase (Pat) has been described in U.S. Pat. Nos. 5,273,894, 5,276,268, and 5,550,318; and gene for bialaphos resistance gene (Bar) has been described in U.S. Pat. Nos. 5,561,236 and 5,646,024, 5,648,477, and 7,112,665. Gene for glutamine synthetase (GS) has been described in U.S. Pat. No. 4,975,372 and European patent application EP 0333033 A1.

E. Resistance/tolerance genes of hydroxy phenyl pyruvate dioxygenase (HPPD) against herbicides isoxazole, diketonitriles, and/or triketones including sulcotrione and mesotrione have been described in U.S. Pat. Nos. 6,268,549 and 6,069,115.

F. Genes for 2,4-D resistance/tolerance. Gene of 2,4-D-monooxygenase has been described in U.S. Pat. Nos. 6,100,446 and 6,153,401. Additional genes for 2,4-D resistance/tolerance are disclosed in US 2009/0093366 (AAD-1) and WO 2007/053482 (AAD-12), the content of which are incorporated by reference in their entireties.

G. Gene of imidazoleglycerol phosphate dehydratase (IGPD) against herbicides imidazole and/or triazole has been described in U.S. Pat. No. 5,541,310. Genes of Dicamba degrading enzymes (oxygenase, ferredoxin, and reductase) against herbicide Dicamba have been disclosed in U.S. Pat. Nos. 7,022,896 and 7,105,724.

H. Genes for herbicides that inhibit photosynthesis, including triazine (psbA and 1s+ genes) or a benzonitrile (nitrilase gene). See e.g., Przibila et al., Plant Cell 3:169 (1991) disclosing transformation of Chlamydomonas with plasmids encoding mutant psbA genes. Nucleotide sequences for nitrilase genes are disclosed in U.S. Pat. No. 4,810,648 and DNA molecules containing these genes are available under ATCC Accession Nos. 53435, 67441, and 67442. Cloning and expression of DNA coding for a glutathione S-transferase is described by Hayes et al., Biochem. J. 285:173 (1992).

The expression of isolated nucleic acids encoding a protein of the present invention can be achieved by operably linking, for example, the DNA or cDNA to a promoter (which is either constitutive or regulatable), followed by incorporation into an expression vector. The vectors can be suitable for replication and integration in either prokaryotes or eukaryotes. Typical expression vectors contain transcription and translation terminators, initiation sequences, and promoters useful for regulation of the expression of the DNA encoding a protein of the present invention. To obtain high level expression of a cloned gene, it is desirable to construct expression vectors which contain, at the minimum, a promoter to direct transcription, a ribosome binding site for translational initiation, and a transcription/translation terminator. One of skill will recognize that modifications can be made to a protein of the present invention without diminishing its biological activity. Some modifications can be made to facilitate the cloning, expression, or incorporation of the targeting molecule into a fusion protein. Such modifications are well known to those of skill in the art and include, for example, a methionine added at the amino terminus to provide an initiation site, or additional amino acids (e.g., poly His) placed on either terminus to create conveniently located purification sequences. Restriction sites or termination codons can also be introduced.

Prokaryotic cells can be used as hosts for expression. Prokaryotes most frequently are represented by various strains of E. coli; however, other microbial strains can also be used. Commonly used prokaryotic control sequences which are defined herein to include promoters for transcription initiation, optionally with an operator, along with ribosome binding site sequences, include such commonly used promoters as the beta lactamase (penicillinase) and lactose (lac) promoter systems (Chang et al., Nature 198:1056 (1977)), the tryptophan (trp) promoter system (Goeddel et al., Nucleic Acids Res. 8:4057 (1980)) and the lambda derived P L promoter and N-gene ribosome binding site (Shimatake et al., Nature 292:128 (1981)). The inclusion of selection markers in DNA vectors transfected in E. coli is also useful. Examples of such markers include genes specifying resistance to ampicillin, tetracycline, or chloramphenicol.

The vector is selected to allow introduction into the appropriate host cell. Bacterial vectors are typically of plasmid or phage origin. Appropriate bacterial cells are infected with phage vector particles or transfected with naked phage vector DNA. If a plasmid vector is used, the bacterial cells are transfected with the plasmid vector DNA. Expression systems for expressing a protein of the present invention are available using Bacillus sp. and Salmonella. See e.g., Palva et al., Gene 22:229-235 (1983); and Mosbach et al., Nature 302:543-545 (1983).

The proteins of this invention, recombinant or synthetic, can be purified to substantial purity by standard techniques well known in the art, including detergent solubilization, selective precipitation with such substances as ammonium sulfate, column chromatography, immunopurification methods, and others. See e.g., R. Scopes, Protein Purification: Principles and Practice, Springer-Verlag: New York (1982); and Deutscher, Guide to Protein Purification, Academic Press (1990). For example, antibodies can be raised to the proteins as described herein. Purification from E. coli can be achieved following procedures described in U.S. Pat. No. 4,511,503. The protein can then be isolated from cells expressing the protein and further purified by standard protein chemistry techniques as described herein.

The present invention provides methods for expressing a plurality of enzyme activities through the use of gene fusion constructs or chimeric DNAs, as well as methods for producing modified gene constructs. In addition, the present invention provides the gene fusion constructs for use in these methods, and the modified gene fusion constructs prepared by these methods. Gene fusion constructs in their simplest form are combinations of nucleic acid sequences encoding enzymatic domains. The constructs can further include nucleic acid sequences that participate in expression of the encoded hybrid protein, such as transcription elements, promoters, termination sequences, introns, and the like. In addition, the constructs can include nucleotide linker sequences such as those described below.

The nucleic acid sequences cojoined to form the gene fusion constructs and modified gene fusion constructs of the present invention can be various forms of deoxyribonucleic acid (for example, genomic DNA, cDNA, sense-strand sequences, antisense-strand sequences, recombinant DNA, shuffled DNA, modified DNA, or DNA analogs). Alternatively, the nucleic acid sequences can be ribonucleic acid (including, but not limited to, genomic RNA, messenger RNA, catalytic RNA, sense-strand sequences, antisense-strand sequences, recombinant RNA, shuffled RNA, modified RNA, or RNA analogs). The nucleic acid sequences incorporated into the fusion constructs of the present invention can also be derived from one or more libraries of nucleic acid sequences.

The gene fusion constructs and modified gene fusion constructs of the present invention can be prepared by a number of techniques known in the art, such as molecular cloning techniques. A wide variety of cloning and in vitro amplification methods suitable for the construction of recombinant nucleic acids, such as expression vectors, are well-known to persons of skill General texts which describe molecular biological techniques useful herein, including mutagenesis, include Berger and Kimmel, Guide to Molecular Cloning Techniques, Methods in Enzymology, volume 152 Academic Press, Inc., San Diego, Calif. (“Berger”); Sambrook, et al., Molecular Cloning—A Laboratory Manual (2nd Ed.), volumes 1-3, Cold Spring Harbor Laboratory, Cold Spring Harbor, N.Y., 1989 (“Sambrook”); and Current Protocols in Molecular Biology, FM. Ausubel, et al., eds., Current Protocols, a joint venture between Greene Publishing Associates, Inc. and John Wiley & Sons, Inc., (supplemented through 2000) (“Ausubel”)).

Examples of techniques sufficient to direct persons of skill through in vitro amplification methods, including the polymerase chain reaction (PCR) the ligase chain reaction (LCR), Qβ-replicase amplification and other RNA polymerase mediated techniques (e.g., NASBA) are found in Berger, Sambrook, and Ausubel, as well as Mullis, et al., (1987) U.S. Pat. No. 4,683,202; PCR Protocols A Guide to Methods and Applications (Innis, et al., eds.) Academic Press Inc. San Diego, Calif. (1990); Arnheim & Levinson (Oct. 1, 1990) Chemical and Engineering News 36-47; The Journal Of NIH Research (1991) 3:81-94; Kwoh, et al., (1989) Proc Natl Acad. Sci. USA 86:1173; Guatelli, et al., (1990) Proc Natl Acad Sci USA 87:1874; Lomell, et al., (1989) J Clin Chem 35:1826; Landegren, et al., (1988) Science 241:1077-1080; Van Brunt (1990) Biotechnology 8:291-294; Wu and Wallace, (1989) Gene 4:560; Barringer, et al., (1990) Gene 89:117, and Sooknanan and Malek (1995) Biotechnology 13:563-564. Improved methods of cloning in vitro amplified nucleic acids are described in U.S. Pat. No. 5,426,039. Improved methods of amplifying large nucleic acids by PCR are summarized in Cheng, et al., (1994) Nature 369:684-685 and the references therein, in which PCR amplicons of up to 40 kb are generated. One of skill will appreciate that essentially any RNA can be converted into a double stranded DNA suitable for restriction digestion, PCR expansion and sequencing using reverse transcriptase and a polymerase.

Detection of the expressed protein in all in vivo systems can be achieved by methods known in the art including, for example, radioimmunoassays, Western blotting techniques, and immunoprecipitation.

As used herein, the phrase “trait” refers to a physiological, morphological, biochemical, or physical characteristic of a plant or particular plant material or cell. In some instances, this characteristic is visible to the human eye, including seed or plant size, or can be measured by biochemical techniques, including detecting the protein, starch, or oil content of seed or leaves, or by observation of a metabolic or physiological process (e.g., by measuring uptake of carbon dioxide), or by the observation of the expression level of a gene or genes (e.g., by employing Northern analysis, RT-PCR, microarray gene expression assays), or reporter gene expression systems, or by agricultural observations including stress tolerance, yield, or pathogen tolerance.

As used herein, the phrase “overexpression” refers to a greater expression level of a gene in a plant, plant cell or plant tissue, compared to expression in a wild-type plant, cell or tissue, at any developmental or temporal stage for the gene. Overexpression can occur when, for example, the genes are under the control of a strong expression signal, including one of the promoters described herein or the cauliflower mosaic virus 35S transcription initiation region known in the art. Overexpression may occur throughout a plant or in specific tissues of the plant, depending on the promoter used.

Enhanced traits of particular interest include those to seed (such as embryo or endosperm), fruit, root, flower, leaf, stem, shoot, seedling or the like, including: enhanced tolerance to environmental conditions including freezing, chilling, heat, drought, water saturation, radiation and ozone; improved tolerance to microbial, fungal or viral diseases; improved tolerance to pest infestations, including insects, nematodes, mollicutes, parasitic higher plants or the like; decreased herbicide sensitivity; improved tolerance of heavy metals or enhanced ability to take up heavy metals; improved growth under poor photoconditions (e.g., low light and/or short day length), or changes in expression levels of genes of interest.

Other phenotype that can be modified relate to the production of plant metabolites, such as variations in the production of taxol, tocopherol, tocotrienol, sterols, phytosterols, vitamins, wax monomers, anti-oxidants, amino acids, lignins, cellulose, tannins, prenyllipids (including chlorophylls and carotenoids), glucosinolates, and terpenoids, enhanced or compositionally altered protein or oil production (especially in seeds), or modified sugar (insoluble or soluble) and/or starch composition. Physical plant characteristics that can be modified include cell development (including the number of trichomes), fruit and seed size and number, yields of plant parts such as stems, leaves, inflorescences, and roots, the stability of the seeds during storage, characteristics of the seed pod (e.g., susceptibility to shattering), root hair length and quantity, internode distances, or the quality of seed coat. Plant growth characteristics that can be modified include growth rate, germination rate of seeds, vigor of plants and seedlings, leaf and flower senescence, male sterility, apomixis, flowering time, flower abscission, rate of nitrogen uptake, osmotic sensitivity to soluble sugar concentrations, biomass or transpiration characteristics, as well as plant architecture characteristics such as apical dominance, branching patterns, number of organs, organ identity, organ shape or size.

Chilling Tolerance: Plants can be enhanced to extend the effective growth range of chilling sensitive crop species by allowing earlier planting or later harvest during a growing season. Chilling tolerance can serve as a model for understanding how plants adapt to water deficit. Both chilling and water stress share similar signal transduction pathways and tolerance/adaptation mechanisms. For example, acclimation to chilling temperatures can be induced by water stress or treatment with abscisic acid. Genes induced by low temperature include dehydrins (or LEA proteins). Dehydrins are also induced by salinity, abscisic acid, water stress and during the late stages of embryogenesis.

Cold Germination: Plants can be enhanced to confer better germination and growth in cold conditions. Genes that would allow germination and seedling vigor in the cold would have highly significant utility in allowing seeds to be planted earlier in the season with a high rate of survival. Genes that confer better survival in cooler climates allow a grower to move up planting time in the spring and extend the growing season further into autumn for higher crop yields. Germination of seeds and survival at temperatures significantly below that of the mean temperature required for germination of seeds and survival of non-transformed plants would increase the potential range of a crop plant into regions in which it would otherwise fail to thrive.

Osmotic Stress, Freezing tolerance, and Drought Tolerance: Plants can be enhanced to confer better tolerance for salt stress, general osmotic stress, drought stress and freezing stress, have the ability to impact whole plant and cellular water availability. Exposure to dehydration may invoke similar survival strategies in plants as does freezing stress, and drought stress may induce freezing tolerance. In addition to the induction of cold-acclimation proteins, strategies that allow plants to survive in low water conditions may include, for example, reduced surface area, or surface oil or wax production. Osmotic stresses may be regulated by specific molecular control mechanisms that include genes controlling water and ion movements, functional and structural stress-induced proteins, signal perception and transduction, and free radical scavenging, and many others. Instigators of osmotic stress may include freezing, drought and high salinity.

In many ways, freezing, high salt and drought have similar effects on plants, not the least of which is the induction of common polypeptides that respond to these different stresses. For example, freezing is similar to water deficit in that freezing reduces the amount of water available to a plant. Exposure to freezing temperatures may lead to cellular dehydration as water leaves cells and forms ice crystals in intercellular spaces. As with high salt concentration and freezing, the problems for plants caused by low water availability include mechanical stresses caused by the withdrawal of cellular water. Thus, the incorporation of transgenes that modify a plant's response to osmotic stress into, for example, a crop or ornamental plant, may be useful in reducing damage or loss.

Heat Stress Tolerance: The germination of many crops is also sensitive to high temperatures. Genes which can provide increased heat tolerance, are generally useful in producing plants that germinate and grow in hot conditions, may find particular use for crops that are planted late in the season, or extend the range of a plant by allowing growth in relatively hot climates. These genes may allow much faster generation times. In a number of species, for example, broccoli, cauliflower, where the reproductive parts of the plants constitute the crop and the vegetative tissues are discarded, it would be advantageous to accelerate time to flowering. Accelerating flowering could shorten crop and tree breeding programs. Additionally, in some instances, a faster generation time would allow additional harvests of a crop to be made within a given growing season.

Flowering Time: By regulating the expression of potential flowering using inducible promoters, flowering can be triggered by application of an inducer chemical. This would allow flowering to be synchronized across a crop and facilitate more efficient harvesting. Such inducible systems could also be used to tune the flowering of crop varieties to different latitudes. At present, species such as soybean and cotton are available as a series of maturity groups that are suitable for different latitudes on the basis of their flowering time (which is governed by day-length). A system in which flowering could be chemically controlled would allow a single high-yielding northern maturity group to be grown at any latitude. In southern regions such plants could be grown for longer periods before flowering is induced, thereby increasing yields. In more northern areas, the induction would be used to ensure that the crop flowers prior to the first winter frosts. In addition, flower structure may have advantageous or deleterious effects on fertility, and could be used, for example, to decrease fertility by the absence, reduction or screening of reproductive components.

Root Development and Morphology: By modifying the structure or development of roots by modifying expression levels of one or more of the presently disclosed genes, plants may be produced that have the capacity to thrive in otherwise unproductive soils. For example, grape roots extending further into rocky soils would provide greater anchorage, greater coverage with increased branching, or would remain viable in waterlogged soils, thus increasing the effective planting range of the crop and/or increasing yield and survival. It may be advantageous to manipulate a plant to produce short roots, as when a soil in which the plant will be growing is occasionally flooded, or when pathogenic fungi or disease-causing nematodes are prevalent.

Seed Development and Germination Rate: Plants can be enhanced for seed development and germination rate, including when the seeds are in conditions normally unfavorable for germination (e.g., cold, heat or salt stress), and may, along with functional equivalogs, thus be used to modify and improve germination rates under adverse conditions.

Fast Growth and/or Plant Size: Plants can be enhanced to accelerate seedling growth, and thereby allow a crop to become established faster. This would minimize exposure to stress conditions at early stages of growth when the plants are most sensitive. Additionally, it can allow a crop to grow faster than competing weed species. Larger plants produce more biomass. For some ornamental plants, the ability to provide larger varieties with these genes or their equivalogs may be highly desirable. More significantly, crop species overexpressing these genes from diverse species would also produce higher yields on larger cultivars, particularly those in which the vegetative portion of the plant is edible.

Overexpression of these genes can confer increased stress tolerance as well as increased biomass, and the increased biomass appears to be related to the particular mechanism of stress tolerance exhibited by these genes. The decision for a lateral organ to continue growth and expansion versus entering late development phases (growth cessation and senescence) is controlled genetically and hormonally, including regulation at an organ size checkpoint. See e.g., Mizukami and Fisher (2000) Proc. Natl. Acad. Sci. 97: 942-47; and Hu et al. Plant Cell 15: 1591. Organ size is controlled by the meristematic competence of organ cells, with increased meristematic competence leading to increased organ size (both leaves and stems). Plant hormones can impact plant organ size, with ethylene pathway overexpression leading to reduced organ size.

Large Seedlings: Plants can be enhanced to produce large seedlings which can be used to produce crops that become established faster. Large seedlings are generally hardier, less vulnerable to stress, and better able to out-compete weed species. Seedlings in which expression of some of the presently disclosed genes may lead to larger cotyledons and/or more developmentally advanced seedlings than control plants. Rapid seedling development made possible by manipulating expression of these genes or their equivalogs is likely to reduce loss due to diseases particularly prevalent at the seedling stage (e.g., damping off) and is thus important for survivability of plants germinating in the field or in controlled environments.

Leaf Morphology and/or Leaf Development: Plants can be enhanced for leaf morphology, including leaf color, leaf size, glossy leaf, and/or leaf thickness. Enhanced chlorophyll and carotenoid levels can also improve yield in crop plants. Lutein, like other xanthophylls such as zeaxanthin and violaxanthin, is an essential component in the protection of the plant against the damaging effects of excessive light. Specifically, lutein contributes, directly or indirectly, to the rapid rise of non-photochemical quenching in plants exposed to high light. Crop plants engineered to contain higher levels of lutein can therefore have improved photo-protection, leading to less oxidative damage and better growth under high light (e.g., during long summer days, or at higher altitudes or lower latitudes than those at which a non-transformed plant would thrive). Additionally, elevated chlorophyll levels increases photosynthetic capacity.

Light Response and/or Shade Avoidance: Plants can be enhanced for response to light as useful for modifying plant growth or development, for example, photomorphogenesis in poor light, or accelerating flowering time in response to various light intensities, quality or duration to which a non-transformed plant would not similarly respond. Examples of such responses that have been demonstrated include leaf number and arrangement, and early flower bud appearances. Elimination of shading responses may lead to increased planting densities with subsequent yield enhancement.

Nutrient Uptake and Utilization: Presently disclosed genes introduced into plants provide a means to improve uptake of essential nutrients, including nitrogenous compounds, phosphates, potassium, and trace minerals. Young plants have a rapid intake of phosphate and sufficient phosphate is important for yield of root crops such as carrot, potato and parsnip. The utilities of presently disclosed genes conferring tolerance to conditions of low nutrients also include cost savings to the grower by reducing the amounts of fertilizer needed, environmental benefits of reduced fertilizer runoff into watersheds; and improved yield and stress tolerance. In addition, by providing improved nitrogen uptake capability, these genes can be used to alter seed protein amounts and/or composition in such a way that could impact yield as well as the nutritional value and production of various food products.

Oxidative Stress Tolerance: Plants can be enhanced to oxidative stress tolerance including oxygen radicals, for example superoxide and peroxide radicals. Generally, plants that have the highest level of defense mechanisms, for example, polyunsaturated moieties of membrane lipids, are most likely to thrive under conditions that introduce oxidative stress (e.g., high light, ozone, water deficit, particularly in combination). One specific oxidizing agent, ozone, has been shown to cause significant foliar injury, which impacts yield and appearance of crop and ornamental plants.

Heavy Metal Tolerance: Heavy metals such as lead, mercury, arsenic, chromium and others may have a significant adverse impact on plant respiration. Plants can be enhanced to confer improved resistance to heavy metals, through, for example, sequestering or reduced uptake of the metals will show improved vigor and yield in soils with relatively high concentrations of these elements. Alternatively, plants can be enhanced an increase in heavy metal uptake, which may benefit efforts to clean up contaminated soils.

Seed Morphology and Number: Plants can be enhanced to alter the size or number of seeds which may have a significant impact on yield, either when the product is the seed itself, or when biomass of the vegetative portion of the plant is increased by reducing seed production. In the case of fruit products, it is often advantageous to modify a plant to have reduced size or number of seeds relative to non-transformed plants to provide seedless or varieties with reduced numbers or smaller seeds. Seed size, in addition to seed coat integrity, thickness and permeability, seed water content and by a number of other components including antioxidants and oligosaccharides, may affect seed longevity in storage. This would be an important utility when the seed of a plant is the harvested crops, as with, for example, peas, beans, nuts, etc. Plants can also be enhanced to modify seed color, which could provide added appeal to a seed product.

Compositions and methods herein may be used for transformation of any plant species, including, but not limited to, monocots and dicots. In some examples, the transformed plant species is a seed crop plant, a grain plant, an oil-seed plant, and/or a leguminous plant.

Plant species transformed in particular examples include, for example and without limitation, crop plants; corn (maize; Zea mays); Brassica sp. (e.g., B. napus, B. rapa, and B. juncea); alfalfa (Medicago sativa); rice (Oryza sativa); rye (Secale cereale); sorghum (Sorghum bicolor, Sorghum vulgare); millet (e.g., pearl millet (Pennisetum glaucum), proso millet (Panicum miliaceum), foxtail millet (Setaria italica), finger millet (Eleusine coracana)); sunflower (Helianthus annuus); safflower (Carthamus tinctorius); wheat (Triticum aestivum); soybean (Glycine max); tobacco (Nicotiana tabacum); potato (Solanum tuberosum); peanut (Arachis hypogaea); cotton (Gossypium barbadense, G. hirsutum, etc.); sweet potato (Ipomoea batatus); cassava (Manihot esculenta); coffee (Coffea spp.); coconut (Cocos nucifera); pineapple (Ananas comosus); citrus trees (Citrus spp.); cocoa (Theobroma cacao); tea (Camellia sinensis); banana (Musa spp.); avocado (Persea americana); fig (Ficus casica); guava (Psidium guajava); mango (Mangifera indica); olive (Olea europaea); papaya (Carica papaya); cashew (Anacardium occidentale); macadamia (Macadamia integrifolia); almond (Prunus amygdalus); sugar beet (Beta vulgaris); sugarcane (Saccharum spp.); oats (Avena sativa); barley (Hordeum vulgare); vegetables; ornamentals; and conifers.

Vegetable plant species transformed in particular examples include, for example and without limitation, tomatoes (Lycopersicon esculentum); lettuce (e.g., Lactuca sativa); green beans (Phaseolus vulgaris); lima beans (Phaseolus limensis); peas (Lathyrus spp.); and members of the genus Cucumis, such as cucumber (C. sativus), cantaloupe (C. cantalupensis), and musk melon (C. melo).

Ornamental plant species transformed in particular examples include, for example and without limitation, azalea (Rhododendron spp.); hydrangea (Macrophylla hydrangea); hibiscus (Hibiscus rosasanensis); roses (Rosa spp.); tulips (Tulipa spp.); daffodils (Narcissus spp.); petunias (Petunia hybrida); carnation (Dianthus caryophyllus); poinsettia (Euphorbia pulcherrima); and chrysanthemum.

Conifer plant species transformed in particular examples include, for example and without limitation, pines, such as loblolly pine (Pinus taeda), slash pine (Pinus eiiiotii), ponderosa pine (Pinus ponderosa), lodgepole pine (Pinus contorta), and Monterey pine (Pinus radiata); Douglas-fir (Pseudotsuga menziesii); Western hemlock (Tsuga canadensis); Sitka spruce (Picea glauca); redwood (Sequoia sempervirens); true firs, such as silver fir (Abies amabilis) and balsam fir (Abies balsamea); and cedars, such as Western red cedar (Thuja plicata) and Alaska yellow-cedar (Chamaecyparis nootkatensis).

While the invention has been described with reference to specific methods and embodiments, it will be appreciated that various modifications and changes may be made without departing from the invention. All publications cited herein are expressly incorporated herein by reference for the purpose of describing and disclosing compositions and methodologies that might be used in connection with the invention. All cited patents, patent applications, and sequence information in referenced websites and public databases are also incorporated by reference.

### EXAMPLES

### Example 1

### Exemplary Field Screening Trials

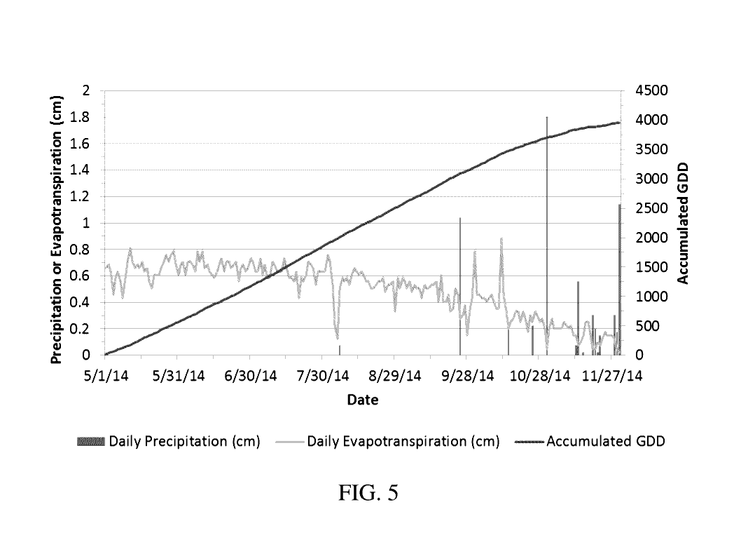

Location 1 and Location 2 are selected for field screening trials because of their low in-season rainfall probability, uniform soil properties, high-quality water availability, and adequate infrastructure. FIG. 5 shows daily precipitation, daily evapotranspiration, and accumulated growing degree day (GDD) information for Location 1 and Location 2 tested. At each location, the soil moisture content of the seedbed is extremely low prior to planting. Using an aboveground sprinkler system, irrigation water is applied to the soil surface for all areas prescribed to be occupied by the field screening trial plots (i.e., pre-irrigation). Roughly 5 to 7 days after pre-irrigation is completed at a site, research plots at that site are planted onto the beds using a 6-row research planter. The seeding rate for all plots is 84,000 plants/ha. All plots are irrigated during the growing season via a drip-tape system, with tape placed in close proximity to each planted row. To insure the absence of water-limiting conditions throughout the growing season, irrigation is done on a weekly basis.

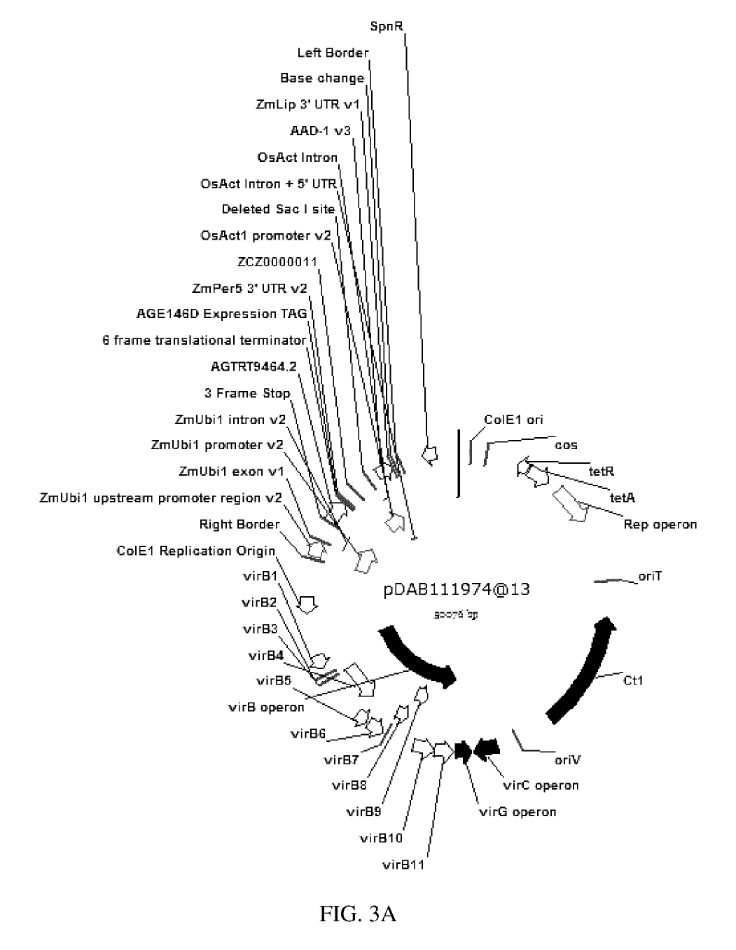

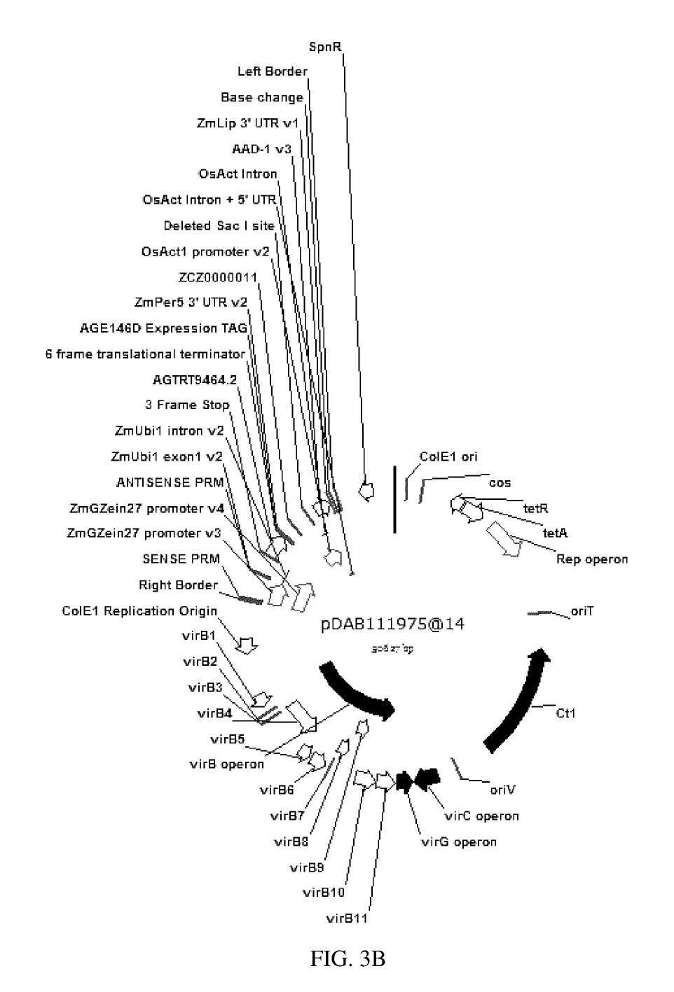

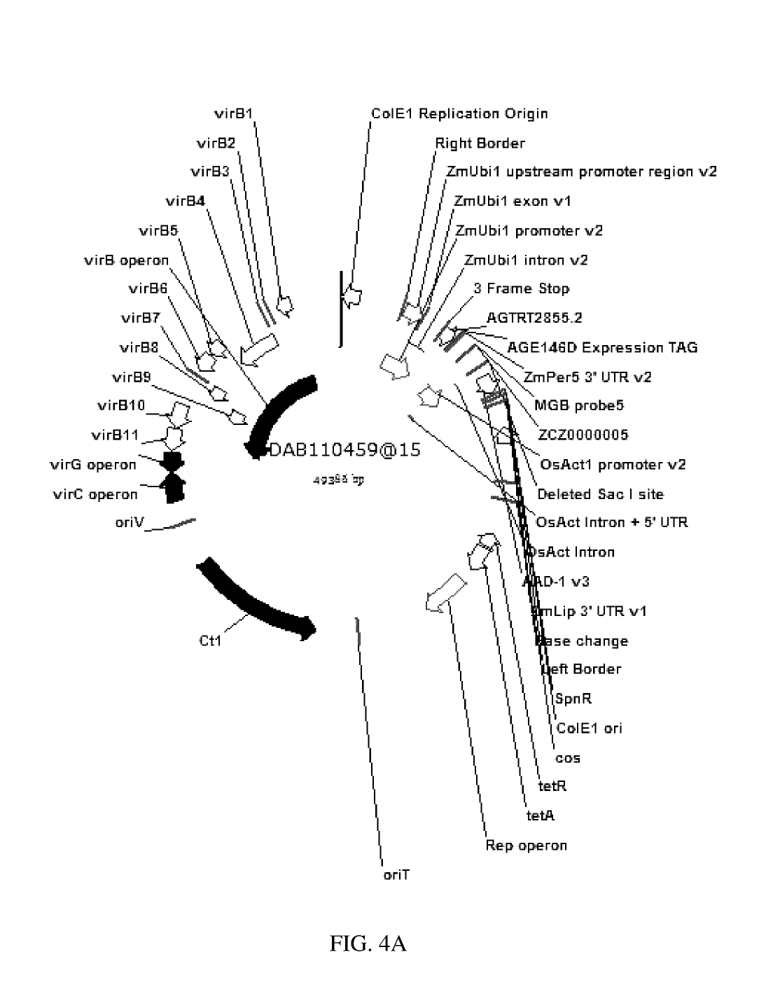

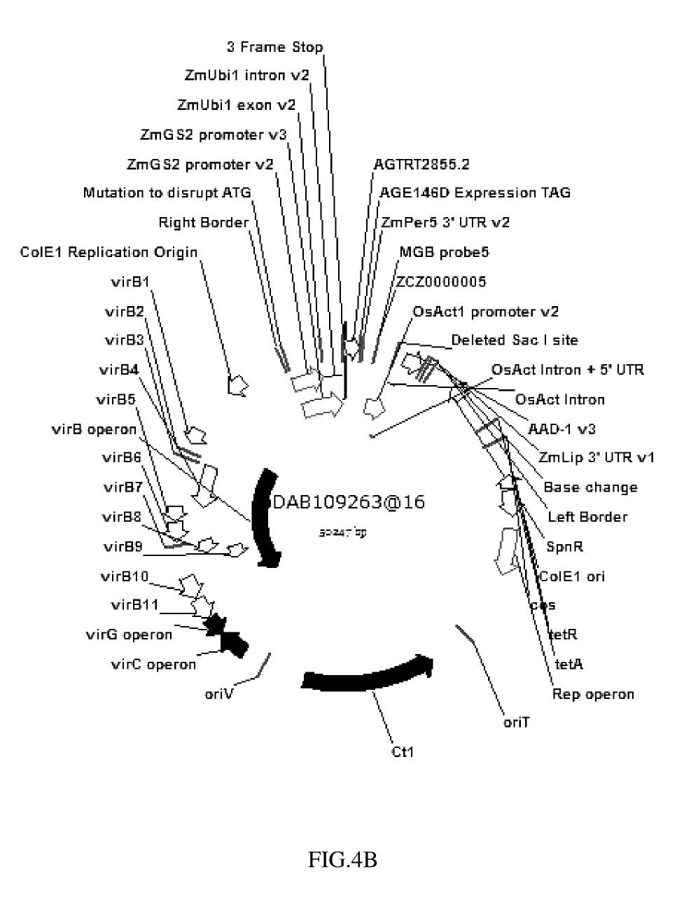

Both the pDAB111974 (i.e., ZmUbi1::BpqPE9; FIG. 3A) and pDAB111975 (i.e., ZmGZein27::BpqPE9; FIG. 3B) constructs (and their respective events) are tested in a B104×LLH37 corn hybrid background. To examine the impact of these constructs and events on key plant metrics, multiple negative controls (i.e., B104×LLH37 hybrid entries without ZmUbi1::BpqPE9 and ZmGZein27::BpqPE9 construct insertions) are also planted at each location.

Seed production of B104 female inbreds containing and not containing events belonging to the pDAB111974 and pDAB111975 constructs is used to assess inbred seed yield potential. Seed production for the field screening trials involved the use of isolated crossing blocks (ICB). B104 female inbreds are intermingled with male inbred tester in a 1:1 female:male row pattern. Multiple planting delays are used to insure synchronization between silk extrusion and pollen shed for the B104 and LLH37 inbreds, respectively. Female rows are detasseled upon tassel emergence to avoid self-pollination. Pollinated ears are monitored regularly for insect and disease symptoms. Once the moisture content of the seeds is at or below 35%, ears in female rows are harvested by hand, dried to proper moisture content, and shelled. The identity of each corn ear is maintained throughout the process.

TABLE 1
Observation numbers for grain yield by location
and nitrogen (N) treatment level

| | |
|---|---|
| Location 1 | Location 2 |

| Entry | High N | Low N | High N | Low N |
|---|---|---|---|---|
| pDAB111974 | 138 | 137 | 97 | 141 |
| pDAB111975 | 134 | 130 | 103 | 141 |
| Negative Control | 294 | 302 | 257 | 336 |
| All | 1381 | 1389 | 1060 | 1320 |

Prior to seed production, event characterization activities are performed across multiple seed production generations (i.e., T₀, T₁, T₁S₁, and T₁S₂). Such activities included leaf sampling, DNA isolation, quantitative real-time polymerase chain reaction (qPCR) analysis, selectable marker screening, RNA-based gene-of-interest (GOI) expression analysis, and GOI zygosity analysis.

Table 1 shows observation numbers for grain yield by location and N treatment level for (i) the pDAB111974 and pDAB111975 constructs and their respective events, (ii) negative control plots, and (iii) all 12 trial constructs (and their associated events) and negative control plots.

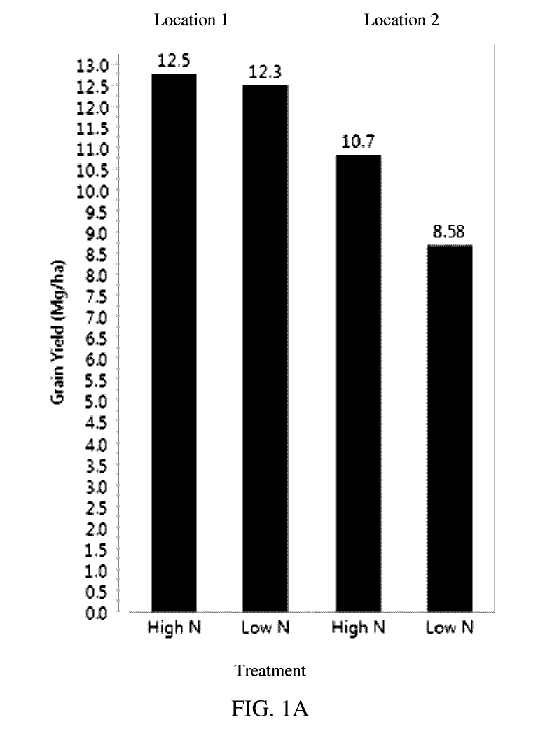

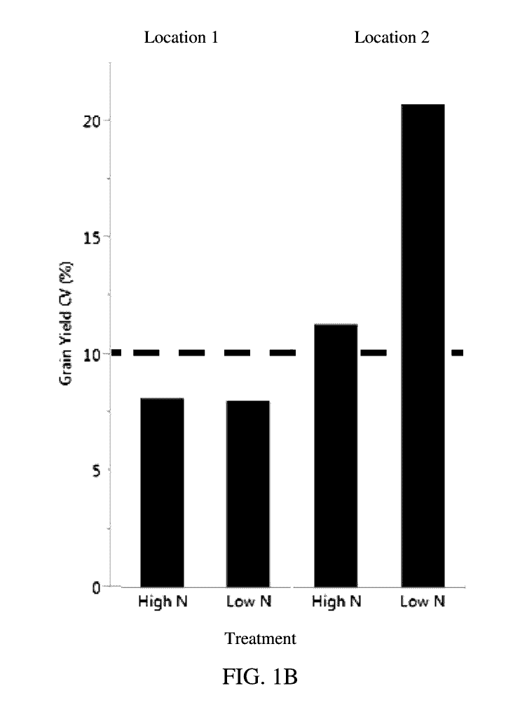

FIG. 1A shows grain yield responses by location and N treatment level when averaged across both constructs and their respective events. Given this experiment's grain yield target of 11 to 12 Mg/ha for high N blocks, both locations with a high N block generally achieved their high N grain yield targets and thus served as good screens for genetic yield potential. FIG. 1B shows grain yield CV values by location and N treatment level at Location 1 and Location 2 tested. Values above 10% are considered undesirably high.

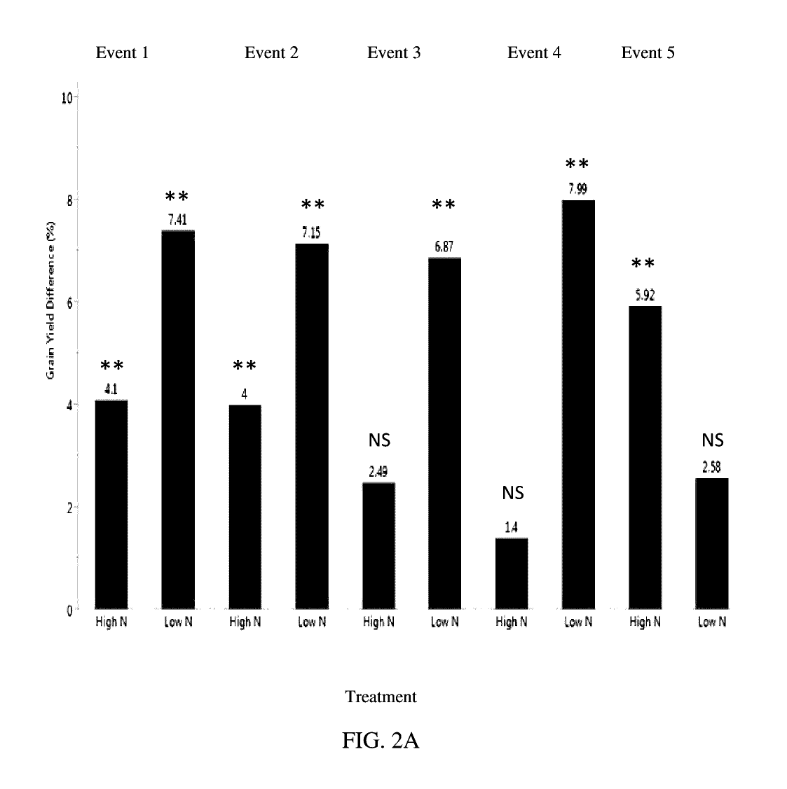

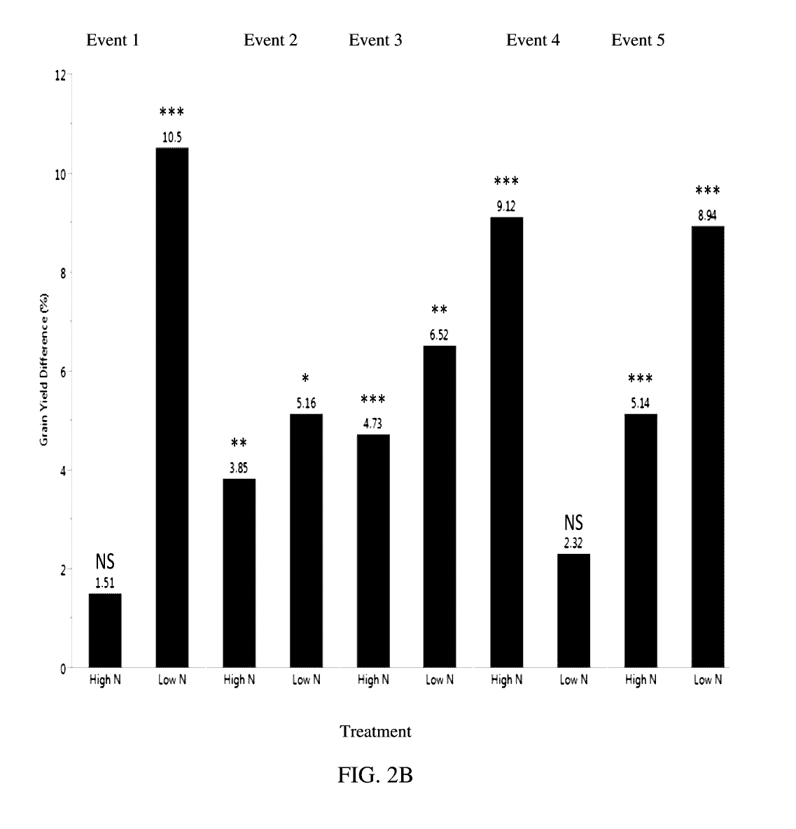

Grain yield differences between five selected events for the pDAB111974 are shown in FIG. 2A. Similarly, grain yield differences between five selected events for the pDAB111975 are shown in FIG. 2B. Thus, we conclude that the constructs pDAB111974 and/or pDAB111975 can significantly increase grain yield under either high N or low N conditions.

### Example 2

### Additional Exemplary Field Screening Trials

Locations 1, 2, and 3 are selected for field screening trials because of their low in-season rainfall probability, uniform soil properties, high-quality water availability, and adequate infrastructure. At each location, the soil moisture content of the seedbed is extremely low prior to planting. Using an aboveground sprinkler system, irrigation water is applied to the soil surface for all areas prescribed to be occupied by the field screening trial plots (i.e., pre-irrigation). Roughly 5 to 7 days after pre-irrigation is completed at a site, research plots at that site are planted onto the beds using a 6-row research planter. The seeding rate for all plots is 84,000 plants/ha. All plots are irrigated during the growing season via a drip-tape system, with tape placed in close proximity to each planted row. To insure the absence of water-limiting conditions throughout the growing season, irrigation is done on a weekly basis.

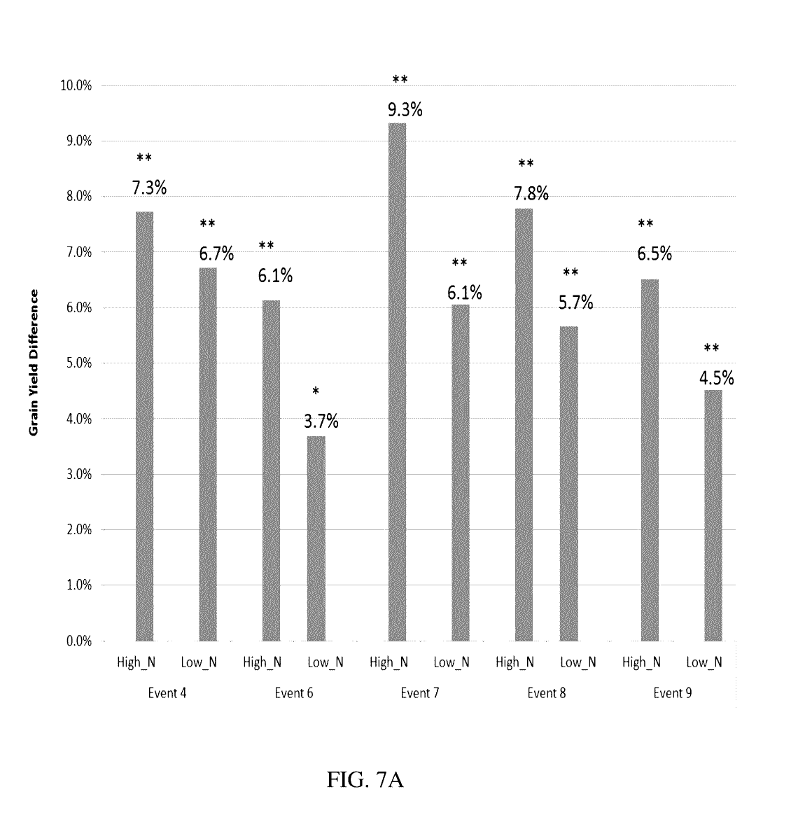

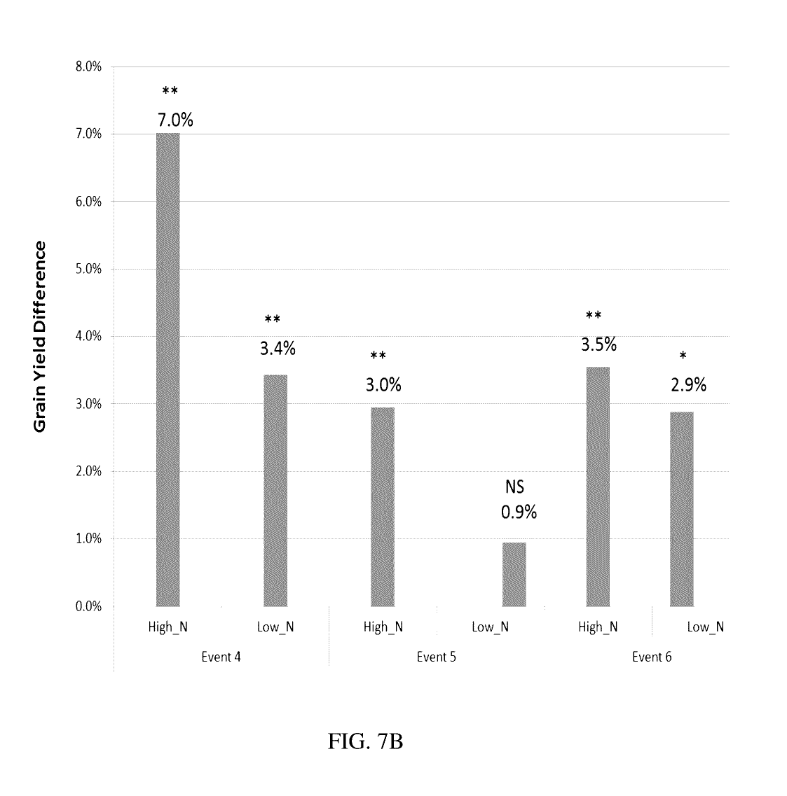

Both the pDAB111974 (i.e., ZmUbi1::BpqPE9; FIG. 7A) and pDAB111975 (i.e., ZmGZein27::BpqPE9; FIG. 7B) constructs (and their respective events) are tested in a B104×LLH37 corn hybrid background. To examine the impact of these constructs and events on key plant metrics, multiple negative controls (i.e., B104×LLH37 hybrid entries without ZmUbi1::BpqPE9 and ZmGZein27::BpqPE9 construct insertions) are also planted at each location.

Seed production of B104 female inbreds containing and not containing events belonging to the pDAB111974 and pDAB111975 constructs are used to assess inbred seed yield potential. Seed production for the field screening trials involved the use of isolated crossing blocks (ICB). B104 female inbreds are intermingled with male inbred tester in a 1:1 female:male row pattern. Multiple planting delays are used to insure synchronization between silk extrusion and pollen shed for the B104 and LLH37 inbreds, respectively. Female rows are detasseled upon tassel emergence to avoid self-pollination. Pollinated ears are monitored regularly for insect and disease symptoms. Once the moisture content of the seeds is at or below 35%, ears in female rows are harvested by hand, dried to proper moisture content, and shelled. The identity of each corn ear is maintained throughout the process.

Prior to seed production, event characterization activities are performed across multiple seed production generations (i.e., T₀, T₁, T₁S₁, and T₁S₂). Such activities included leaf sampling, DNA isolation, quantitative real-time polymerase chain reaction (qPCR) analysis, selectable marker screening, RNA-based gene-of-interest (GOI) expression analysis, and GOI zygosity analysis.

Table 2 shows observation numbers for grain yield by location and N treatment level for (i) the pDAB111974 and pDAB111975 constructs and their respective events, (ii) negative control plots, and (iii) all 12 trial constructs (and their associated events) and negative control plots.

TABLE 2
Observation numbers for grain yield by
location and nitrogen (N) treatment level

| | Location 1 | | Location 2 | | Location 3 | |
|---|---|---|---|---|---|---|
| Entry | High N | Low N | High N | Low N | High N | Low N |
| Negative Control | 81 | 84 | 49 | 39 | 79 | 39 |
| pDAB111974 | 208 | 203 | 137 | 100 | 207 | N.A. |
| pDAB111975 | 269 | 265 | 166 | 138 | 259 | 226 |
| Total | 558 | 552 | 352 | 277 | 545 | 265 |

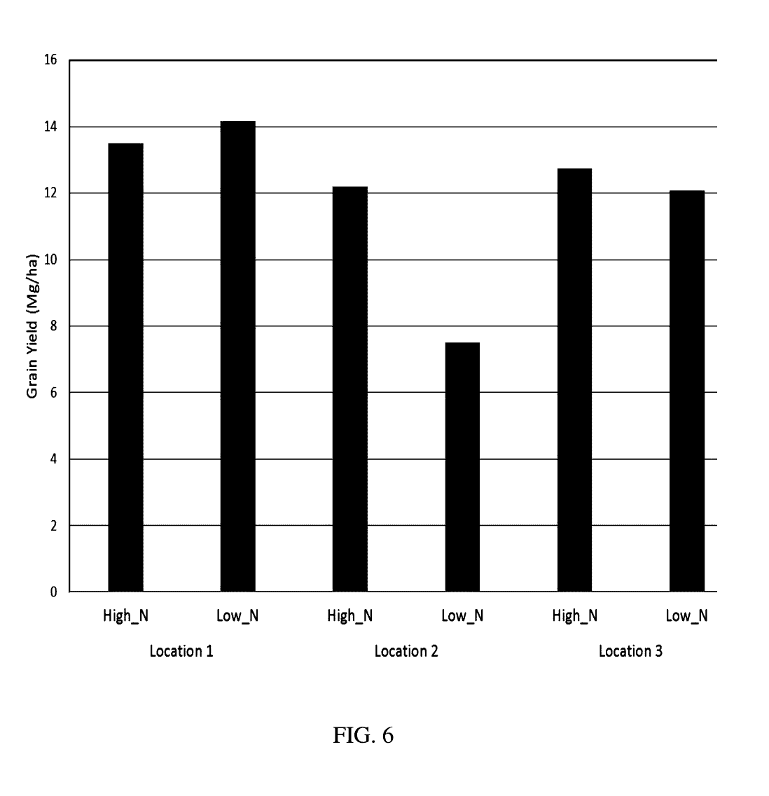

FIG. 6 shows grain yield responses by location and N treatment level when averaged across both constructs and their respective events. Given this experiment's grain yield target of 11 to 12 Mg/ha for high N blocks, both locations with a high N block generally achieved their high N grain yield targets and thus served as good screens for genetic yield potential. The low N treatment did not significantly affect yields in at these locations.

Grain yield differences between five selected events for the pDAB111974 are shown in FIG. 2A. Grain yields showed significant increases in these events compared with isogenic non-transgenic hybrids. Event 4 showed significant yield increases in both 2014 and 2015.

Similarly, grain yield differences between three selected events for the pDAB111975 are shown in FIG. 2B. Grain yields showed significant increases in these event compared with isogenic non-transgenic hybrids. Events 4 and 5 showed significant yield increases in both 2014 and 2015.

Thus, events from constructs pDAB111974 and/or pDAB111975 can significantly increase grain yield under either high N or low N conditions.

We claim:

1. A plant transformation vector comprising a nucleic acid encoding a polypeptide having at least 80% sequence identity to SEQ ID NO: 2 or 4.

2. The plant transformation vector of claim 1, wherein the nucleic acid is operably linked to a constitutive promoter.

3. The plant transformation vector of claim 1, wherein the nucleic acid has at least 80% sequence identity to SEQ ID NO: 1 or 3.

4. A nucleic acid construct for transgenic plants, comprising,
(a) a polynucleotide sequence encoding a polypeptide having at least 80% sequence identity to SEQ ID NO: 2 or 4; and
(b) one or more control sequences for driving expression of the polynucleotide sequence in the transgenic plants;
wherein the polynucleotide sequence is codon optimized for expression in the transgenic plants.

5. The nucleic acid construct of claim 4, wherein the plants are monocotyledons plants.

6. The nucleic acid construct of claim 4, wherein the plants are dicotyledons plants.

7. The nucleic acid construct of claim 4, wherein the nucleic acid construct is stably transformed into the transgenic plants.

8. The nucleic acid construct of claim 4, wherein the nucleic acid construct comprises a binary vector for Agrobacterium-mediated transformation.

9. The nucleic acid construct of claim 4, wherein the nucleic acid construct comprises a selectable marker.

10. The nucleic acid construct of claim 4, wherein the polynucleotide sequence has at least 80% identity to SEQ ID NO: 1 or 3.

11. The nucleic acid construct of claim 4, wherein the one or more control sequences comprise a viral sequence.

12. The nucleic acid construct of claim 4, wherein the one or more control sequences comprise a plant promoter.

13. A method for producing a transgenic plant, the method comprising:
introducing into the plant a heterologous nucleic acid encoding a polypeptide having at least 80% sequence identity to SEQ ID NO: 1 or 3.

14. The method of claim 13, wherein the heterologous nucleic acid is introduced into the plant by Agrobacterium-mediated transformation.

15. A method for enhancing crop yield, comprising,
(a) transforming a plant cell with the plant transformation vector of claim 1 or nucleic acid construct of claim 4;
(b) regenerating the transformed plant cell into a transgenic plant; and
(c) planting the transgenic plant in a crop field.

16. The method of claim 15, wherein there is statistically significant improvement of at least one parameter selected from the group consisting of grain yield per hectare (GYH), grain yield per plant (GPP), averaged grain size (AGS), kernel number per plant (KPP), and averaged kernel weight (AKW) as compared to negative control plant.

17. A transgenic plant generated from the method of claim 13 or 15.

18. The transgenic plant of claim 17, wherein the plant is Zea mays or Glycine max.

19. A plant comprising a heterologous nucleic acid encoding a polypeptide having at least 80% sequence identity to a polypeptide selected from the group consisting of SEQ ID NO: 2 or 4.

20. The plant of claim 19, wherein there is statistically significant improvement of at least one parameter selected from the group consisting of grain yield per hectare (GYH), grain yield per plant (GPP), averaged grain size (AGS), kernel number per plant (KPP), and averaged kernel weight (AKW) as compared to negative control plant.

21. A process for producing a plant, plant seed, or progeny thereof, comprising,
(a) transforming a plant cell with the plant transformation vector of claim 1 or nucleic acid construct of claim 4;
(b) growing a plant from the transformed plant cell until the plant produces seed; and
(c) harvesting the seed from the plant.

22. A seed harvested from the plant produced by the process of claim 21.

23. A genetically transformed plant or seed, characterized in that its genome has been transformed to contain the plant transformation vector of claim 1 or nucleic acid construct of claim 4.

24. The genetically transformed plant or seed of claim 23, characterized in that there is statistically significant improvement of at least one parameter selected from the group consisting of grain yield per hectare (GYH), grain yield per plant (GPP), averaged grain size (AGS), kernel number per plant (KPP), and averaged kernel weight (AKW) as compared to its non-transgenic parent plant or seed.
